(19) United States
(12) Patent Application Publication
(10) Pub. No.: US 2024/0238300 A1
(43) Pub. Date: Jul. 18, 2024

(54) TREATMENT METHODS FOR SUBJECTS WITH CANCER HAVING AN ABERRATION IN EGFR AND/OR HER2

(71) Applicants: Jill KREMER, Princeton, NJ (US); TAIHO PHARMACEUTICAL CO., LTD., Tokyo (JP)

(72) Inventors: Kei OGUCHI, Tsukuba-shi (JP); Jill KREMER, Princeton, NJ (US); Karim BENHADJI, Princeton, NJ (US)

(73) Assignee: TAIHO PHARMACEUTICAL CO., LTD., Tokyo (JP)

(21) Appl. No.: 18/563,058

(22) PCT Filed: May 23, 2022
(86) PCT No.: PCT/US2022/030500

§ 371 Date: Nov. 21, 2023

Related U.S. Application Data
(60) Provisional application No. 63/192,351, filed on May 24, 2021.

(51) Int. Cl. A61K 31/519; A61P 35/00

(52) U.S. Cl. CPC A61K 31/519; A61P 35/00

(57) ABSTRACT

A method of treating a subject with cancer having an aberration in EGFR and/or HER2, whereby the subject is administered 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

### CROSS-REFERENCE TO RELATED APPLICATIONS

The present application is based on and claims the benefit of priority to U.S. Application No. 63/192,351, filed May 24, 2021. The entire content of this application is incorporated herein by reference.

### BACKGROUND OF THE INVENTION

### Field of the Invention

The present invention relates to methods of treating cancers harboring an aberration in EGFR and/or HER2.

### Description of the Related Art

The erythroblastosis oncogene B (ErbB) receptor family includes four receptor tyrosine kinases: EGFR/HER1/ErbB1, HER2/ErbB2, HER3/ErbB3, and HER4/ErbB4. These 4 receptors are activated via homodimerization, heterodimerization, and possibly higher-order oligomers, resulting in autophosphorylation of tyrosine residues within the cytoplasmic domain of the receptors, thereby activating a variety of signaling pathways, including Ras and phosphoinositide 3-kinase (PI3K). With the exception of ErbB4, aberrant activation of the ErbB receptors contributes to oncogenesis. In particular, aberrations in EGFR and/or HER2 are found in many cancer types, and therefore much research has been focused on identifying inhibitors capable of regulating the kinase activity of these receptors. However, cancers harboring certain types of aberrations within the ErbB receptor family have proven difficult to treat, leaving patients harboring these cancer types with no or limited treatment options.

HER2 exon 20 insertion mutations are oncogenic alterations found in non-small lung cancer (NSCLC) patients, which accounts for approximately 4% of NSCLC. However, approved HER2 tyrosine kinase inhibitors (TKIs) (afatinib, lapatinib, and neratinib) have limited efficacy for those patients. See Robichaux J P, Elamin Y Y, Tan Z, Carter B W. Zhang S, Liu S, et al. Mechanisms and clinical activity of an EGFR and HER2 exon 20-selective kinase inhibitor in non-small cell lung cancer. Nat Med. 2018 May; 24(5):638-646. In addition, EGFR exon 20 insertion mutations, which account for approximately 4-13% of all NSCLC EGFR mutations, also generally have low sensitivity to EGFR-TKI monotherapies, for example: (i) afatinib (Yasuda H, Park E, Yun C H, Sng N J, Lucena-Araujo A R, Yeo W L, et al. Structural, biochemical, and clinical characterization of epidermal growth factor receptor (EGFR) exon 20 insertion mutations in lung cancer. Sci Transl Med. 2013 December; 5(216):216ra177; Yang J C, Sequist L V, Geater S L, Tsai C M, Mok T S, Schuler M, et al. Clinical activity of afatinib in patients with advanced non-small-cell lung cancer harbouring uncommon EGFR mutations: a combined post-hoc analysis of LUX-Lung 2, LUX-Lung 3, and LUX-Lung 6. Lancet Oncol. 2015 July; 16(7):830-8); (ii) gefitinib (Wu J Y, Wu S G, Yang C H, Gow C H, Chang Y L, Yu C J, et al. Lung cancer with epidermal growth factor receptor exon 20 mutations is associated with poor gefitinib treatment response. Clin Cancer Res. 2008 August; 14(15):4877-82; Yasuda H, Park E, Yun C H, Sng N J, Lucena-Araujo A R, Yeo W L, et al. Structural, biochemical, and clinical characterization of epidermal growth factor receptor (EGFR) exon 20 insertion mutations in lung cancer. Sci Transl Med. 2013 December; 5(216):216ra177); (iii) erlotinib (Yasuda H, Park E, Yun C H, Sng N J, Lucena-Araujo A R, Yeo W L, et al. Structural, biochemical, and clinical characterization of epidermal growth factor receptor (EGFR) exon 20 insertion mutations in lung cancer. Sci Transl Med. 2013 December; 5(216):216ra177; Naidoo J., Sima C S, Rodriguez K, Busby N, Nafa K, Ladanyi M, et al. Epidermal growth factor receptor exon 20 insertions in advanced lung adenocarcinomas: Clinical outcomes and response to erlotinib. Cancer 2015 September; 15; 121(18):3212-3220); (iv) osimertinib, and (v) dacomitinib (Robichaux J P, Elamin Y Y, Tan Z, Carter B W, Zhang S, Liu S, et al. Mechanisms and clinical activity of an EGFR and HER2 exon 20-selective kinase inhibitor in non-small cell lung cancer. Nat Med. 2018 May; 24(5):638-646).

Several reports have also indicated that approximately 25-40% of NSCLC and approximately 15-30% of breast cancers could develop brain metastases. See Witzel I, Oliveira-Ferrer L, Pantel K, Müller V, Wikman H. Breast cancer brain metastases: biology and new clinical perspectives. Breast Cancer Res. 2016 January; 18(1):8; Abdallah S M, Wong A. Brain metastases in non-small-cell lung cancer: are tyrosine kinase inhibitors and checkpoint inhibitors now viable options? Curr Oncol. 2018 June; 25 (Suppl 1): S103-S114. Patients with primary tumors that metastasize to the brain have even more limited treatment options, as many inhibitors cannot efficiently cross the blood-brain barrier (BBB) or are substrates for efflux transporters expressed at high levels at the BBB such as P-glycoprotein (P-gp) or ATP-binding cassette super-family G member 2 (ABCG2; also known as breast cancer resistance protein, BCRP). Indeed, there are no brain penetrable TKIs approved for NSCLC with HER2/EGFR exon 20 insertions.

Further, EGFR overexpression has been found in a variety of human tumors, such as glioblastoma (GBM), often as a consequence of gene amplification. EGFR overexpression is frequently associated with various kinds of mutation, one of the most common mutations being the extracellular domain mutation, EGFR variant III (EGFRvIII). See Gan, H K, Cvrljevic, A N, and Johns, T G, The epidermal growth factor receptor variant III (EGFRvIII): where wild things are altered. FEBS J, 2013; 280: 5350-5370. This mutation leads to a deletion of exons 2-7 of the EGFR gene and renders the mutant receptor incapable of binding any known ligand. Despite this, EGFRvIII displays low-level constitutive signaling that is augmented by reduced internalization and downregulation. Aberrant EGFRvIII signaling has been shown to be important in driving tumor progression and often correlates with poor prognosis. See An Z, Aksoy O, Zheng T, Fan Q W, Weiss W A. Epidermal growth factor receptor and EGFRvIII in glioblastoma: signaling pathways and targeted therapies. Oncogene. 2018 March; 37(12):1561-1575. There are no approved drugs to date targeting GBM caused by EGFR genetic alterations.

Still further, EGFR-positive and/or HER2-positive tumors that also contain certain oncogenic gene fusions may result in aberrant ErbB-mediated pathway activation. One example of which are fusions involving the neuregulin-1 gene (NRG1). NRG1 fusions result in irregular expression of the epidermal growth factor (EGF)-like domain of NRG1 on the cell surface, which serves as a ligand for ErbB3 and induces the formation of heterodimers, most frequently ErbB2-ErbB3, but also with EGF receptor (EGFR; ErbB1) and ErbB4. This leads to pathologic activation of the phosphoinositide 3-kinase-protein kinase B (PI3K-Akt), mitogen-activated protein kinase (MAPK), and other signaling pathways, resulting in abnormal cell proliferation. Treatment for patients with NRG1 fusion-driven tumors, as a result of constitutive ErbB-mediated pathway activation, remain an unmet medical need.

Still further, the treatment of recurrent or refractory cancers is a major challenge across virtually all cancer types, such as, inter alia, breast cancer, gastric cancer, and ovarian cancer. Patients that have EGFR-positive and/or HER2-positive tumors, but nonetheless fail to respond to, or relapse from, treatment using approved EGFR/HER2 inhibitors are often left with few remaining treatment options. In some cases, the refractory nature of the cancer is itself induced through drug resistance that is acquired during one or more initial treatment regimens, for example treatment with EGFR/HER2 inhibitor(s), leading to tumor invasion, metastasis, and poor clinical outcomes. There is thus a substantial unmet medical need for new therapies in patients with recurrent or refractory forms of cancer, particularly those whom have received prior therapy.

In view of the forgoing, there exists a need for new treatment methods in patients with cancers harboring having an aberration in EGFR and/or HER2, particularly those with EGFR/HER2 exon 20 insertion mutations, primary tumors which have metastasized to the brain, brain cancers with EGFR aberrations, cancers driven from aberrant ErbB-mediated pathway activation due to oncogenic gene fusions, and recurrent or refractory EGFR-positive and/or HER2-positive cancers.

### SUMMARY OF THE INVENTION

Accordingly, it is an aspect of the present invention to provide methods of treating a subject with a cancer having an aberration in EGFR and/or HER2.

It is another aspect of the present invention to provide methods of treating a subject with a cancer having an EGFR exon 20 insertion mutation and/or a HER2 exon 20 insertion mutation, including those which have metastasized.

It is another aspect of the present invention to provide methods of treating a subject with a cancer having an EGFR exon 20 insertion mutation and/or a HER2 exon 20 insertion mutation, which has metastasized to the brain.

It is another aspect of the present invention to provide methods of treating a brain cancer harboring an EGFR amplification and/or an EGFRvIII mutation.

It is another aspect of the present invention to provide methods of treating cancers with oncogenic gene fusions, such as NRG1 fusions or others driven from aberrant ErbB-mediated pathway activation.

It is yet another aspect of the present invention to provide methods of treating recurrent or refractory HER2-positive cancers.

These and other aspects, which will become apparent during the following detailed description, have been achieved by the inventors' finding that 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof, a brain-penetrable pan-ErbB inhibitor, is active in and can be used for treating cancers harboring EGFR and/or HER2 aberrations such as those listed above. The present invention has the following aspects:

(1) A method of a treating a subject with a cancer having at least one aberration in EGFR and/or HER2, the method comprising administering to the subject an effective amount 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof. This compound having the formula below is referred to as Compound (1):

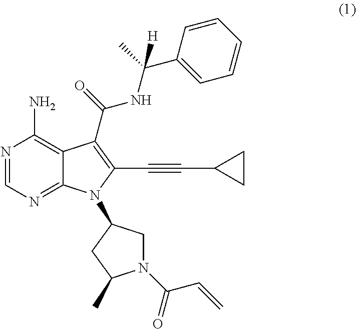 [SMILES: C=CC(=O)N1C[C@H](n2c(C#CC3CC3)c(C(=O)N[C@H](C)c3ccccc3)c3c(N)ncnc32)C[C@@H]1C]

(2) The method of (1), wherein the cancer is a solid tumor.

(3) The method of (1) or (2), wherein the cancer is at least one selected from the group consisting of lung cancer, breast cancer, gastric cancer, bladder cancer, and biliary cancer.

(4) The method of any one of (1) to (3), wherein the cancer has metastasized to the brain of the subject.

(5) The method of any one of (1) to (4), wherein the cancer has an EGFR amplification/overexpression and/or a HER2 amplification/overexpression.

(6) The method of any one of (1) to (5), wherein the cancer has an EGFR mutation and/or a HER2 mutation.

(7) The method of any one of (1) to (6), wherein the cancer has an EGFR exon 20 insertion mutation and/or a HER2 exon 20 insertion mutation.

(8) The method of any one of (1) to (7), wherein the cancer has an EGFR exon 20 insertion mutation.

(9) The method of any one of (1) to (8), wherein the cancer has a HER2 exon 20 insertion mutation.

(10) The method of any one of (7) to (9), wherein the cancer has metastasized to the brain of the subject.

(11) The method of any one of (1) to (10), wherein the cancer is non-small cell lung cancer.

(12) The method of (1), wherein the cancer has an EGFR amplification and/or an EGFRvIII mutation.

(13) The method of (12), wherein the cancer is glioblastoma.

(14) The method of (1), wherein the cancer having an aberration in EGFR and/or HER2 is a cancer harboring an aberration in NRG1.

(15) The method of (14), wherein the cancer harboring an aberration in NRG1 is an NRG1 fusion-driven cancer harboring an NRG1 fusion.

(16) The method of (14), wherein the cancer harboring an aberration in NRG1 is at least one selected from the group consisting of lung cancer, breast cancer, colorectal cancer, pancreatic cancer, biliary cancer, ovarian cancer, and gastric cancer.

(17) The method of (15), wherein the NRG1 fusion-driven cancer is at least one selected from the group consisting of lung cancer, breast cancer, colorectal cancer, pancreatic cancer, biliary cancer, and ovarian cancer.

(18) The method of any one of (1) to (17), wherein the cancer is unresectable.

(19) The method of any one of (1) to (18), wherein the cancer is a recurrent or refractory cancer.

(20) The method of any one of (1) to (19), wherein the cancer is a recurrent or refractory HER2-positive cancer.

(21) The method of (20), wherein the subject with the recurrent or refractory HER2-positive cancer has previously undergone a treatment regimen with a HER2 inhibitor prior to administering 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

(22) The method of (21), wherein the recurrent or refractory HER2-positive cancer acquired resistance to or intractability from the treatment regimen with the HER2 inhibitor.

(23) The method of (21) or (22), wherein the HER2 inhibitor is a HER2 tyrosine kinase inhibitor.

(24) The method of (23), wherein the HER2 tyrosine kinase inhibitor is at least one selected from the group consisting of afatinib, lapatinib, neratinib, and tucatinib.

(25) The method of (21) or (22), wherein the HER2 inhibitor is an anti-HER2 antibody or drug conjugate thereof.

(26) The method of (25), wherein the anti-HER2 antibody or drug conjugate thereof is at least one selected from the group consisting of trastuzumab, trastuzumab emtansine, pertuzumab, margetuximab, and trastuzumab deruxtecan.

(27) The method of any one of (20) to (26), wherein the subject with the recurrent or refractory HER2-positive cancer has previously undergone at least two treatment regimens with at least two different HER2 inhibitors prior to administering 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

(28) The method of any one of (20) to (27), wherein the recurrent or refractory HER2-positive cancer is recurrent or refractory HER2-positive breast cancer or recurrent or refractory HER2-positive gastric cancer.

(29) The method of any one of (1) to (28), wherein the subject is determined to have the aberration in EGFR and/or HER2 prior to administering 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

(30) The method of any one of (1) to (29), wherein the 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered orally to the subject.

(31) The method of any one of (1) to (30), wherein the 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered to the subject once per day (QD).

(32) The method of any one of (1) to (30), wherein the 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered to the subject twice per day (BID).

(33) The method of any one of (1) to (32), wherein about 5 to about 480 mg of 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered to the subject per day.

(34) The method of any one of (1) to (333), wherein about 15 to about 240 mg of 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered to the subject per day.

(35) The method of any one of (1) to (34), wherein the 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered daily to the subject for at least 28 days.

(36) An antitumor agent for treating a subject with a cancer having an aberration in EGFR and/or HER2, the antitumor agent comprising 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

(37) Use of 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof in the treating of a subject with a cancer having an aberration in EGFR and/or HER2.

### BRIEF DESCRIPTION OF THE DRAWINGS

The foregoing paragraphs have been provided by way of general introduction, and are not intended to limit the scope of the following claims. The described embodiments, together with further advantages, will be best understood by reference to the following detailed description when considered in conjunction with the accompanying drawing, wherein:

FIG. 1 illustrates the antitumor effect of Compound (1) or poziotinib in nude mice bearing NCI-N87 human gastric carcinoma xenograft tumors (HER2 amplification/overexpression), as a function of tumor volume (TV); results are shown as the mean±standard error (SE) (n=6 animals/group).

FIG. 2 illustrates the antitumor effect of Compound (1) or poziotinib in nude mice bearing MCF10A_HER2/insYVMA_v human mammary epithelial xenografts (a human mammary epithelial cell line engineered to express the HER2 exon 20 insertion mutation A775_G776insYVMA), as a function of tumor volume (TV); results are shown as the mean±standard error (SE) (n=6 animals/group).

FIG. 3 illustrates the antitumor effect of Compound (1) or poziotinib in nude mice bearing intracranial NCI-N87-luc (engineered to stably express the luciferase gene using the HER2-overexpressing NCI-N87 human gastric carcinoma cell line) xenograft tumors, as a function of total flux (photons per second; p/s); results are shown as the mean standard error (SE) (n=8 animals/group).

FIGS. 4A and 4B illustrate the plasma concentration-time profiles of Compound (1) in linear scale (FIG. 4A) and semi-logarithmic scale (FIG. 4B) after single oral administration to male nude mice; data are shown as the mean±SD (N=3/group).

FIGS. 5A and 5B illustrate the plasma concentration-time profiles of Compound (1) in linear scale (FIG. 5A) and semi-logarithmic scale (FIG. 5B) after multiple oral administrations for 7 days to male nude mice; data are shown as the mean±SD (N=3/group).

FIGS. 6A and 6B illustrate the antitumor effect and the body weight change by treatment with Compound (1) in a refractory model after BALB/cAJcl-mu/mu male mice implanted with NCI-N87 human gastric carcinoma (HER2 overexpressing)(1.0×10⁷ cells in 100 μL of PBS/mouse, subcutaneous) had relapsed from treatment with a combination of trastuzumab (T-mab) and pertuzumab (P-mab), as a function of tumor volume (TV) *Dunnet's test: p<0.001 (FIG. 6A), and body weight change (BWC) (FIG. 6B).

FIGS. 7A and 7B illustrate the antitumor effect and the body weight change by treatment with Compound (1) in a refractory model after BALB/cAJcl-nu/mu male mice implanted with NCI-N87 human gastric carcinoma (HER2 overexpressing)(1.0×10⁷ cells in 100 μL of PBS/mouse, subcutaneous) had relapsed from treatment with intravenous trastuzunab emtansine (T-DM1) once every 3 weeks at a dosage of 10 mg/kg/day, as a function of tumor volume (TV) (FIG. 7A), and body weight change (BWC) (FIG. 7B).

FIGS. 8A and 8B illustrate the antitumor effect and the body weight change by treatment with Compound (1), compared to lapatinib and tucatinib, in a refractory tumor model in which BALB/cAJ1-nu/mu male mice had been passaged two times with tumor fragments, as a function of tumor volume (TV), Dunnet's test: *p<0.05, ***p<0.001, n.s. no significant change (FIG. 8A), and body weight change (BWC) (FIG. 8B); the tumor fragments being obtained as NCI-N87 human gastric carcinoma (HER2 overexpressing) which had acquired resistance to trastuzumab emtansine (T-DM1) via continuous exposure to intravenous T-DM1 once every 3 weeks at a dosage of 10 mg/kg/day for 126 days.

FIGS. 9A and 9B illustrate the antitumor effect and the body weight change by treatment with Compound (1), compared to trastuzumab deruxtecan (DS-8201a), in a T-DM1 refractory tumor model, as a function of tumor volume (TV), Dunnet's test: ***p<0.001, n.s. no significant change (FIG. 9A), and body weight change (BWC) (FIG. 9B); in the T-DM1 refractory tumor model, a mono-clonal cell line was implanted into BALB/cAJ1-mu/mu male mice (8.0×10⁶ cells in 100 μL of PBS/mouse, subcutaneous), which was derived from tumor fragments of NCI-N87 human gastric carcinoma (HER2 overexpressing) which had acquired resistance to trastuzunab emtansine (T-DM1) via continuous exposure to intravenous T-DM1 once every 3 weeks at a dosage of 10 mg/kg/day for 126 days.

FIG. 10 illustrates the growth inhibitory effect of Compound (1) on MDA-MB-175VII cells (human breast ductal carcinoma which expresses the DOC4-NRG1 fusion protein): results are shown as the mean±standard error (SE) from 3-4 independent experiments.

FIGS. 11A and 11B illustrate the antitumor effect and the body weight change by treatment with Compound (1) with three dosing schedules ((i) QD, (ii) intermittent, and (iii) 4 days-on/3 days-off) in nude mice bearing intracranial NCI-N87-luc (engineered to stably express the luciferase gene using the HER2-overexpressing NCI-N87 human gastric carcinoma cell line) xenograft tumors, as a function of total flux (photons per second: p/s); results are shown as the mean±standard error (SE) (n=7 animals/group).

### DETAILED DESCRIPTION OF THE PREFERRED EMBODIMENTS

Compound (1) is a novel, selective, potent, and orally available brain penetrable pan-Avian (ErbB) inhibitor which is active against EGFR/HER2-positive cell lines. Compound (1) is described in US2021/0024530, Japanese Patent Application No. 2020-121520, Japanese Patent Application No. 2020-121525, and Japanese Patent Application No. 2020-121733, the contents of which are incorporated herein by reference in their entirety.

Compound (1) can be used directly (free form) or in the form of a pharmaceutically acceptable salt. The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. The pharmaceutically acceptable salt of Compound (1) is not particularly limited, and examples thereof include addition salts with inorganic acids such as hydrochloric acid, hydrobromic acid, sulfuric acid, sulfamic acid, phosphoric acid, nitric acid, and the like; organic acids such as acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid, isethionic acid, and the like; salts with alkali metals such as potassium, sodium, and the like; salts with alkaline earth metals such as calcium, magnesium, and the like; and salts with organic bases such as ammonium salts, ethylamine salts, alginate, and the like. The pharmaceutically acceptable salts can be synthesized by conventional chemical methods, generally by reacting Compound (1) with a stoichiometric amount or sub-stoichiometric amount (e.g., 0.5 eq) of the appropriate base or acid in water or in an organic solvent (e.g., ether, ethyl acetate, ethanol, isopropanol, or acetonitrile), or in a mixture of the two.

Compound (1) or a pharmaceutically acceptable salt thereof may be in the form of a “solvate”, which refers to a physical association of a referenced compound with one or more solvent molecules, whether organic or inorganic. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. Solvate encompasses both solution phase and isolable solvates. Exemplary solvent molecules which may form the solvate include, but are not limited to, water, methanol, ethanol, n-propanol, isopropanol, n-butanol, isobutanol, tert-butanol, ethyl acetate, glycerin, acetone, and the like.

Compound (1) can exist in a crystal form type II with 1 eq. fumaric acid (monofumarate) that exhibits an X-ray powder diffraction spectrum containing at least three characteristic peaks at diffraction angles (2θ±0.2°) selected from 5.5°, 6.8°, 9.3°, 13.4°, 15.3°, 16.3°, 18.5°, 19.8°, 22.0°, and 24.5°. Compound (1) can exist in a crystal form type II (free form) that exhibits an X-ray powder diffraction spectrum containing at least three characteristic peaks at diffraction angles (2θ±0.2°) selected from 8.3°, 14.8°, 17.3°, 18.0°, 19.1°, 20.3°, 21.0°, 22.5°. 23.0°, and 26.2°. Compound (1) can exist in a crystal form type I (free form) that exhibits an X-ray powder diffraction spectrum containing at least three characteristic peaks at diffraction angles (2θ±0.2°) selected from 9.9°, 11.7°, 13.2°, 18.8°, and 20.8°. Compound (1) can exist in a crystal form type V with fumaric acid that exhibits an X-ray powder diffraction spectrum containing at least three characteristic peaks at diffraction angles (2θ±0.2°) selected from 6.9°, 13.7°, 21.1°, 23.6°, and 26.5°. Compound (1) can exist in a crystal form type I with 1 eq. fumaric acid that exhibits an X-ray powder diffraction spectrum containing at least three characteristic peaks at diffraction angles (2θ±0.2°) selected from 6.4°, 10.3°, 12.8°, 20.7°, 23.4°, and 26.6°. Particular preference is given to the monofumarate type II crystal form of Compound (1). A crystal meeting any of these criteria shows good stability, excellent oral absorbability, high chemical purity, are non-hygroscopic, and is suitable for mass production. Exemplary methods which can be used for synthesizing Compound (1), as well as those methods which can be used for preparing various crystal forms of Compound (1) are described hereinafter in the Examples section, and also in Japanese Patent Application No. 2020-121520, the contents of which are incorporated herein by reference in its entirety.

The terms “treat”, “treating”, or the “treatment” of cancers in the present disclosure includes any effect, e.g., lessening, reducing, modulating, stabilizing, ameliorating or eliminating, that results in the improvement of the condition, disease, disorder, and the like, or ameliorating a symptom thereof. Specifically, these terms may refer to: (1) a stabilization, reduction (e.g., by more than 10%, 20%, 30%, 40%, 50%, preferably by more than 60% of the population of cancer cells and/or tumor size as compared to prior to administration), or elimination of the cancer cells, (2) inhibiting cancerous cell division and/or cancerous cell proliferation, (3) relieving to some extent (or, preferably, eliminating) one or more symptoms associated with a pathology related to or caused in part by unregulated or aberrant cellular division, (4) an increase in disease-free, relapse-free, progression-free, and/or overall survival, duration, or rate, (5) a decrease in hospitalization rate, (6) a decrease in hospitalization length, (7) eradication, removal, or control of primary, regional and/or metastatic cancer, (8) a stabilization or reduction (e.g., by at least 10%. 20%, 30%, 40%, 50%, 60%, 70%, preferably at least 80% relative to the initial growth rate) in the growth of a tumor or neoplasm, (9) an impairment in the formation of a tumor, (10) a reduction in mortality, (11) an increase in the response rate, the durability of response, or number of patients who respond or are in remission, (12) the size of the tumor is maintained and does not increase or increases by less than 10%, preferably less than 5%, preferably less than 4%, preferably less than 2%, (13) a decrease in the need for surgery (e.g., colectomy, mastectomy), and/or (14) preventing or reducing the metastasis of cancer cells.

The cancers which can be treated herein are EGFR-positive and/or HER2-positive cancers. “EGFR-positive” means a cancer in which an EGFR protein and/or an EGFR gene is detected/detectable, and likewise, “HER2-positive” means a cancer in which a HER2 protein and/or a HER2 gene is detected/detectable. The EGFR protein/gene and/or the HER2 protein/gene may be detected/detectable as wild-type or in altered form (e.g., mutated).

Examples of types of cancers which can be treated include, but are not limited to, glandular tumors, carcinoid tumors, undifferentiated carcinomas, angiosarcoma, adenocarcinoma, gastrointestinal cancers (e.g., colorectal cancers (“CRC”) including colon cancer and rectal cancer, biliary cancers including gall bladder cancer and bile duct cancer (cholangiocarcinoma), anal cancer, esophageal cancer, gastric (stomach) cancer, gastrointestinal carcinoid tumor(s), gastrointestinal stromal tumor(s) (“GIST”), liver cancer, duodenal cancer and small intestine cancer), lung cancers (e.g., non-small cell lung cancer (“NSCLC”), squamous-cell lung carcinoma, large-cell lung carcinoma, small cell lung carcinoma, invasive mucinous adenocarcinoma, mesothelioma and other lung cancers such as bronchial tumors and pleuropulmonary blastoma), urological cancers (e.g., kidney (renal) cancer, transitional cell cancer (“TCC”) of kidney, TCC of the renal pelvis and ureter (“PDQ”), bladder cancer, urethral cancer and prostate cancer), head and neck cancers (e.g., eye cancer, retinoblastoma, intraocular melanoma, hypopharyngeal cancer, pharyngeal cancer, laryngeal cancer, laryngeal papillomatosis, metastatic squamous neck cancer with occult primary, sinonasal squamous cell carcinoma (SNSCC), oral (mouth) cancer, lip cancer, throat cancer, oropharyngeal cancer, esthesioneuroblastoma, nasal cavity and paranasal sinus cancer, nasopharyngeal cancer, and salivary gland cancer), endocrine cancers (e.g., thyroid cancer, parathyroid cancer, multiple endocrine neoplasia syndromes, thymoma and thymic carcinoma, pancreatic cancers including pancreatic ductal adenocarcinoma (“PDAC”), pancreatic neuroendocrine tumors and islet cell tumors), breast cancers (extrahepatic ductal carcinoma in situ (“DCIS”), lobular carcinoma in situ (“LCIS”), triple negative breast cancer, and inflammatory breast cancer), male and female reproductive cancers (e.g., cervical cancer, ovarian cancer, endometrial cancer, uterine sarcoma, uterine cancer, vaginal cancer, vulvar cancer, gestational trophoblastic tumor (“GTD”), extragonadal germ cell tumor, extracranial germ cell tumor, germ cell tumor, testicular cancer and penile cancer), brain and nervous system cancers (e.g., astrocytomas, brain stem glioma, brain tumor, glioblastoma (GBM), craniopharyngioma, central nervous system (“CNS”) cancer, chordomas, ependymoma, embryonal tumors, neuroblastoma, paraganglioma, atypical teratoid, oligodendroma, oligodendroastrocytoma, oligodendroglioma, anaplastic oligodendroastrocytoma, ganglioglioma, central neurocytoma, medulloblastoma, germinoma, meningioma, neurilemmoma, GH secreting pituitary adenoma, PRL-secreting pituitary adenoma, ACTH-secreting pituitary adenoma, nonfunctional pituitary adenoma, hemangioblastoma, and epidermoid tumor), skin cancers (e.g., basal cell carcinoma (“BCC”), squamous cell skin carcinoma (“SCC”), Merkel cell carcinoma and melanoma), tissue and bone cancers (e.g., soft-tissue sarcoma, rhabdomyosarcoma, fibrous histiocytoma of bone, Ewing sarcoma, malignant fibrous histiocytoma of bone (“MFH”), osteosarcoma and chondrosarcoma), cardiovascular cancers (e.g., heart cancer and cardiac tumors), appendix cancers, childhood and adolescent cancers (e.g., adrenocortical carcinoma childhood, midline tract carcinoma, hepatocellular carcinoma (“HCC”), hepatoblastoma and Wilms' tumor) and viral-induced cancers (e.g., HHV-8 related cancers (Kaposi sarcoma) and HIV/AIDS related cancers).

Cancers also suitable for treatment may include, but are not limited to, hematological and plasma cell malignancies (e.g., cancers that affect blood, bone marrow and/or lymph nodes) such as multiple myeloma, leukemias and lymphomas, myelodysplastic syndromes and myeloproliferative disorders. Leukemias include, without limitation, acute lymphoblastic leukemia (“ALL”), acute myelogenous (myeloid) leukemia (“AML”), chronic lymphocytic leukemia (“CLL”), chronic myelogenous leukemia (“CML”), acute monocytic leukemia (“AMoL”), hairy cell leukemia, and/or other leukemias. Lymphomas include, without limitation, Hodgkin's lymphoma and non-Hodgkin's lymphoma (“NHL”). In some embodiments, NHL is B-cell lymphomas and/or T-cell lymphomas. In some embodiments, NHL includes, without limitation, diffuse large B-cell lymphoma (“DLBCL”), small lymphocytic lymphoma (“SLL”), chronic lymphocytic leukemia (“CLL”), mantle cell lymphoma (“MCL”), Burkitt's lymphoma, cutaneous T-cell lymphoma including mycosis fungoides and Sezary syndrome, AIDS-related lymphoma, follicular lymphoma, lymphoplasmacytic lymphoma (Waldenstrom's macroglobulinemia (“WM”)), primary central nervous system (CNS) lymphoma, central nervous system malignant lymphoma, and/or other lymphomas.

Compound (1) or its pharmaceutically acceptable salt forms also possesses high brain penetrability. Therefore, the cancers which can be treated also include metastatic brain tumors, for example, brain metastases of any of the above cancer types, e.g., lung cancers, breast cancers, gastric (stomach) cancer, bladder cancer, or biliary cancers including gall bladder cancer and bile duct cancer, etc., which have metastasized to the brain.

The treatment methods of the present disclosure are particularly useful in the treatment of solid cancers such as lung cancer (e.g., non-small cell lung cancer (“NSCLC”), invasive mucinous adenocarcinoma, etc.), breast cancer (e.g., extrahepatic ductal carcinoma in situ (“DCIS”), lobular carcinoma in situ (“LCIS”), etc.), gastric cancer, bladder cancer, biliary cancer, brain cancer (e.g., glioblastoma), colorectal cancer, pancreatic cancer, and ovarian cancer, as well as any of the above which have metastasized to the brain.

The methods disclosed herein may also be used as a tumor-agnostic treatment for malignancies having an aberration in EGFR and/or HER2, for example, where EGFR and/or HER2 aberrations are found in the form of constitutive ErbB-mediated pathway activation due to aberrant ligand binding, for example NRG1 fusion-driven tumors.

Subjects harboring one or more aberrations in EGFR and/or HER2 are candidates for treatment herein. EGFR and/or HER2 aberrations are key oncogenic drivers that cause activation of a variety of signaling pathways, including Ras and phosphoinositide 3-kinase (PI3K), which in turn contributes to various tumorigenic processes.

In the present disclosure, an “aberration” in EGFR and/or HER2 refers to gain-of-function changes such as protein overexpression, gene amplification (e.g., copy-number alterations), activating gene mutation/activating protein mutation (e.g., insertion, point, or deletion mutations), activating chromosomal translocation/insertion/inversion, activating gene rearrangement or gene fusion (a subset of gene rearrangements), misregulation/dysregulation, and the like, including combinations thereof, that result in or contributes to cancer formation and/or development. For example, cancers harboring EGFR and/or HER2 aberrations may express (i) amplified or overexpressed EGFR and/or HER2, including those of the wild-type variety; (ii) constitutively activated EGFR and/or HER2 (including those of the wild-type variety) due to aberrant ligand binding, such as through an aberrancy in NRG1, e.g., NRG1 fusion-driven binding; (iii) altered EGFR and/or HER2, for example, via activating gene and/or protein mutation(s); or (iv) a combination of aberrations, for example where EGFR and/or HER2 is/are amplified/overexpressed and altered e.g., via mutation(s) in the EGFR gene and/or protein.

Unless specified otherwise, any reference to EGFR amino acid sequence information is based on human wild-type EGFR isoform a, which is accessible from the National Center for Biotechnology Information (NCBI) Protein Database as Accession No. NP_005219.2, P00533.2, etc. Unless specified otherwise, any reference to HER2 amino acid sequence information is based on human wild-type HER2 isoform a, which is accessible from the National Center for Biotechnology Information (NCBI) Protein Database as Accession No. NP_004439.2, NM_004448, etc.

Isoforms of EGFR and HER2 are also known by those of ordinary skill in the art, and the present disclosure also encompasses those isoforms. With regard to alterations (e.g., mutations) in EGFR discussed herein, it should be understood that the alteration in the isoform may be located in a different position from the position identified for EGFR due to deletion or insertion of an amino acid(s) in the isoform, but that the alteration in the isoform nevertheless corresponds to the position identified for EGFR. Likewise, with regard to alterations (e.g., mutations) in HER2 discussed herein, it should be understood that the alteration in the isoform may be located in a different position from the position identified for HER2 due to deletion or insertion of an amino acid(s) in the isoform, but that the alteration in the isoform nevertheless corresponds to the position identified for HER2.

Aberrations in EGFR may be in the form of gene amplification and/or protein overexpression.

Aberrations in EGFR may be in the form of one or more mutations in the EGFR protein. EGFR mutations may be located in the tyrosine kinase domain of EGFR, including, but are not limited to, one or more of: exon 18 (in the region of 688-728); exon 19 (in the region of 729-761); exon 20 (in the region of 762-823); and exon 21 (in the region of 824-875).

EGFR exon 18 mutations may include, but are not limited to, point mutations such as E709X or G719X (where X is an arbitrary amino acid), exemplified by E709K, E709A, E709G, G719A, G719S, and G719C, deletion mutations, and deletion insertion mutations, for example deletion of glutamic acid at position 709 and threonine at position 710 and insertion of aspartic acid (DelE709_T710insD), and the like.

EGFR exon 19 mutations may include, but are not limited to, “classical” Exon 19 deletion mutations of at least three amino acid residues, as well as deletion insertion mutations, for example DelE746_A750 (deletion of glutamic acid at position 746 to alanine at position 750), DelL747_P753insS (deletion of leucine at position 747 to proline at position 753 and insertion of serine), DelE746_T751insA, DelE746_S752insD, DelL747_T751, DelL747_A750insP, and the like.

EGFR exon 20 mutations may include, but are not limited to, point mutations such as T790M, S768I, V769M, and H773R, deletion mutations, and insertion mutations. In particular, compound (1) or its pharmaceutically acceptable salts have been found to be surprisingly active in cancers harboring one or more EGFR exon 20 insertion mutations. EGFR exon 20 insertion mutations are found with relatively high prevalence in non-small lung cancer (NSCLC) as well as sinonasal squamous cell carcinoma (SNSCC), are associated with de novo resistance to current clinically available EGFR inhibitors, and are therefore preferred targets for treatment herein.

EGFR exon 20 insertion mutations may be heterogeneous in-frame insertions of between 1-7 amino acids (indicated as “insX”) across a span of about 15 amino acids (D761-C775) in exon 20, for example D761_E762insX (insertion of between 1-7 amino acid residues “X” in between aspartic acid at position 761 and glutamic acid at position 762), A763_Y764insX, Y764_V765insX, V765_M766insX, A767_S768insX, S768_V769insX. V769_D770insX, D770_N771insX, N771_P772insX, P772_H773insX, H773_V774insX, and V774_C775insX. Also included are deletion insertion mutations, such as DelD770insX (deletion of aspartic acid at position 770 and insertion of 1-7 amino acids “X”) and DelN771insX (Simon Vyse and Paul H. Huang, Targeting EGFR exon 20 insertion mutations in non-small cell lung cancer. Signal Transduct Target Ther. 2019 Mar. 8; 4:5). Specific examples of EGFR exon 20 insertion mutations include, but are not limited to, A763_Y764insFQEA, Y764_V765insHH, A767_S768insASV, S768dupSVD, V769_D770insASV, D770_N771insNPG, D770_N771insGL, D770_N771insSVD, D770_N771insSVG, DelD770insGY, DelD770insVG, N771_P772insH, N771_P772insV, DelN771insGY, DelN77linsTH, P772_H773insDNP, P772_H773insPNP, H773_V774insNPH, H773_V774insPH, H773_V774insAH, H773_V774insH, and V774_C775insHV (Takayuki Kosaka et al. Response Heterogeneity of EGFR and HER2 Exon 20 Insertions to Covalent EGFR and HER2 Inhibitors. Cancer Res. 2017 May; 77(10):2712-2721).

A preferred embodiment of the present disclosure involves treating a subject with a cancer, specific mention being made to non-small lung cancer (NSCLC), harboring an EGFR exon 20 insertion mutation, particularly in cases where such cancers which have metastasized to the brain.

EGFR exon 21 mutations may include, but are not limited to, point mutations such as L858X and L861X (where X is an arbitrary amino acid), such as the “classical” exon 20 activating mutation L858R, as well as L833V, H835L, L838V, A839T, K846R, and L861Q.

Exemplary EGFR proteins which contain multiple mutations, and can be treated herein, may include, but are not limited to, DelE746_A750/T790M and T790M/L858R.

Mutations in the EGFR protein may be those set forth in Japanese Patent Application No. 2020-121525, the contents of which are incorporated herein by reference in their entirety.

Aberrations in EGFR may also be in the form of gene amplification and/or one or more genetic alterations in EGFR, such as EGFR gene rearrangements, and resultant mutations in EGFR in which particular exons or exon parts are deleted. Examples of which include, but are not limited to, EGFR variant I (EGFRvI; deletion of N-terminal part), EGFR variant II (EGFRvII; deletion of exons 14 and 15), EGFR variant III (EGFRvIII; deletion of exons 2-7), EGFR variant IV (EGFRvIV; deletion of exons 25-27), and EGFR variant V (EGFRvV; deletion of exons 25-28).

Of these, gene rearrangements resulting in expression of EGFRvIII is found with high prevalence in glioblastoma (GBM), with expression being identified in other cancers as well, such as breast carcinoma, and lung carcinoma (e.g., NSCLC). EGFRvIII results from the in-frame deletion of 801 base pairs spanning exons 2-7, with this deletion removing 267 amino acids from the extracellular domain, thereby creating a junction site between exons 1 and 8 and a new glycine residue. These alterations confer enhanced tumorgenicity, such as increased rates of proliferation, reduced apoptosis, increased angiogenesis, and increased invasiveness compared to unaltered EGFR, mediated by several downstream signaling pathways, including PI3K/Akt, Ras/Raf/MAPK, signal transducer and activator of transcriptase 3 (Stat3), and nuclear factor kappa-B (NF-κB).

Given the high prevalence of EGFR amplification and EGFRvIII mutations in GBM, the lack of approved drugs targeting GBM harboring these EGFR genetic alterations, and the high brain penetrability of compound (1) or its pharmaceutically acceptable salts, a preferred embodiment of the present disclosure involves treating a subject with GBM harboring an EGFR amplification and/or an EGFRvIII mutation.

Aberrations in HER2 may be in the form of gene amplification and/or protein overexpression.

Aberrations in HER2 may be in the form of one or more mutations in the HER2 protein. HER2 mutations may be located in the tyrosine kinase domain of HER2, including, but are not limited to, one or more of: exon 19 (in the region of 736-769); exon 20 (in the region of 770-831); exon 21 (in the region of 832-883); and exons 22-31 (in the region of 884-1255).

HER2 exon 19 mutations may include, but are not limited to, point mutations such as L755X, I767X, D769X (where X is an arbitrary amino acid), exemplified by L755S, L755P, I767M, D769H, D769N, and D769Y, and deletion mutations such as DelL755_T759, and the like.

HER2 exon 20 mutations may include, but are not limited to, point mutations such as G776X and V777X (where X is an arbitrary amino acid), exemplified by G776V, G776S, V777L, and V777M, deletion mutations, and insertion mutations. In particular, compound (1) or its pharmaceutically acceptable salts have been found to be surprisingly active in cancers harboring one or more HER2 exon 20 insertion mutations. HER2 exon 20 insertion mutations are found in a variety of cancer types, such as inter alia, lung cancer (e.g., NSCLC) and breast cancer, however, treatment of HER2 exon 20 insertions remains a clinical challenge-approved HER2 TKIs afatinib, lapatinib, and neratinib have limited efficacy in this patient population. HER2 exon 20 insertion mutants are thus preferred targets for treatment herein.

HER2 exon 20 insertion mutations may be heterogeneous in-frame insertions of between 1-7 amino acids in exon 20, for example A775_G776insX (insertion of between 1-7 amino acid residues “X” in between alanine at position 775 and glycine at position 776), duplication insertion mutations such as Y772dupYVMA (duplication of the YVMA sequence starting at the tyrosine at position 772), and deletion insertion mutations, such as DelG776insX (deletion of glycine at position 776 and insertion of 1-7 amino acids “X”). Specific examples of HER2 exon 20 insertion mutations include, but are not limited to, Y772dupYVMA, DelM774insWLV, A775_G776insYVMA, A775_G776insSVMA, A775_G776insI, DelG776insVC, DelG776insLC, G778_S779insCPG, V777_G778insGSP, G778dupGSP, and P780_Y78linsGSP (Takayuki Kosaka et al. Response Heterogeneity of EGFR and HER2 Exon 20 Insertions to Covalent EGFR and HER2 Inhibitors. Cancer Res. 2017 May; 77(10):2712-2721; Jacqulyne P Robichaux et al. Pan-Cancer Landscape and Analysis of ERBB2 Mutations Identifies Poziotinib as a Clinically Active Inhibitor and Enhancer of T-DM1 Activity. Cancer Cell. 2019 October; 36(4):444-457).

A preferred embodiment of the present disclosure involves treating a subject with a cancer, specific mention being made to non-small lung cancer (NSCLC), harboring a HER2 exon 20 insertion mutation, particularly in cases where such cancers which have metastasized to the brain.

HER2 exon 21 mutations may include, but are not limited to, point mutations such as V8421 and R868W.

HER2 exon 22-31 mutations may include, but are not limited to, R896C, E1021Q, A1057V, V1128I, and N1219S.

Aberrations in HER2 may be in the form of one or more mutations in the transmembrane or extracellular domain region of HER2, examples of which include, but are not limited to, S310X (where X is an arbitrary amino acid) exemplified by S310F and S310Y, R678X (where X is an arbitrary amino acid) exemplified by R678Q and R678C, as well as G309A, P122L, G222C, 1655V, S305C, H470Q, 1263T, and A293T.

Mutations in the HER2 protein may be those set forth in Japanese Patent Application US2021/0024530, the contents of which are incorporated herein by reference in their entirety.

Aberrations in EGFR and/or HER2 may also be in the form of constitutively activated EGFR and/or HER2 (including those of the wild-type variety) due to aberrant ligand binding. One example of which is cancers having an aberration in NRG1. An “aberration” in NRG1 refers to gain-of-function changes such as protein overexpression, gene amplification (e.g., copy-number alterations), activating gene mutation/activating protein mutation (e.g., insertion, point, or deletion mutations), activating chromosomal translocation/insertion/inversion, activating gene rearrangement or gene fusion (a subset of gene rearrangements), misregulation/dysregulation, and the like, including combinations thereof, that result in or contributes to cancer formation and/or development.

For example, aberrations in NRG1 may be in the form of NRG1 overexpression. NRG1 overexpression has been found to be associated with aggressive tumor features and poor prognosis in gastric cancer patients (Yun, S. et al. Clinical significance of overexpression of NRG1 and its receptors, HER3 and HER4, in gastric cancer patients; Gastric Cancer (2018) 21:225-236).

Another example of which is cancers harboring oncogenic gene fusions in NRG1. NRG1 gene fusions result in irregular expression of the epidermal growth factor (EGF)-like domain of NRG1 on the cell surface, which serves as a ligand for ErbB3 (HER3) and induces the formation of heterodimers, most frequently ErbB2-ErbB3, but also with EGF receptor (EGFR; ErbB1) and ErbB4. This leads to pathologic activation of the phosphoinositide 3-kinase-protein kinase B (PI3K-Akt), mitogen-activated protein kinase (MAPK), and other signaling pathways, resulting in abnormal cell proliferation. This binding event may take place in an autocrine or juxtacrine fashion. Alternatively, in some cases, the EGF-like domain of NRG1 can be cleaved from its surface tether where it binds to ErbB3 (HER3) in a paracrine fashion. In any event, NRG1 fusion-positive cancers cause aberrant activation of EGFR and/or HER2, and thus in a preferred embodiment, the present disclosure involves treating a subject with an NRG1 fusion-driven tumor.

Cancers driven by one or more aberrations in NRG1 may include, but are not limited to, lung cancer, breast cancer, colorectal cancer, pancreatic cancer, biliary cancer, ovarian cancer, and gastric cancer.

Cancers driven by NRG1 gene fusions may include, but are not limited to, lung cancer, breast cancer, colorectal cancer, pancreatic cancer, biliary cancer, and ovarian cancer.

NRG1 fusions may be formed from various fusion partners (listed below as X in X-NRG1), the selection of fusion partner is not particularly limiting. Examples of NRG1 fusions include, but are not limited to, DOC4-NRG1, CD74-NRG1, SLC3A2-NRG1, SDC4-NRG1, RBPMS-NRG1, WRN-NRG1, VAMP2-NRG1, ATP1B1-NRG1, ROCK1-NRG1, RALGAPA1-NRG1, TNC-NRG1, MDK-NRG1, DIP2B-NRG1, MRPL13-NRG1, DPYSL2-NRG1, PARP8-NRG1, ITGB1-NRG1, POMK-NRG1, APP-NRG1, CDH6-NRG1, ATP1B1-NRG1, and CLU-NRG1 (J. Laskin et al. NRG1 fusion-driven tumors: biology, detection, and the therapeutic role of afatinib and other ErbB-targeting agents. Ann Oncol. 2020 December; 31(12):1693-1703).

Other growth factors that activate ErbB receptors that may lead to aberrant ErbB-mediated constitutive pathway activation include, but are not limited to, EGF, TGF-α, HB-EGF, amphiregulin, betacellulin, epigen, and epiregulin. Thus cancers harboring activating aberrations in any of these growth factors that result in aberrant activation of ErbB family receptors and downstream ErbB-mediated signaling pathways, contributing to oncogenesis, may be treated herein.

While cancers at various stages and resectabilities may respond to the disclosed treatment, the methods herein may be particularly useful in the treatment of unresectable, advanced (stage III) and metastatic (stage IV) disease, “recurrent,” and “refractory” cancers-cancer that heretofore has failed to respond to medical treatment. “Recurrent” cancers are cancers that have recurred (come back), usually after a period of time during, which the cancer could not be detected. The cancer may come back to the same place as the original (primary) tumor or to another place in the body. “Refractory” cancers may present as resistance/intractability from the start, or the acquisition of resistance/intractability by the cancer cells during the course of prior therapy, and thus can include relapsed cancer that responds initially to treatment, but returns, often in a more aggressive/resistant form. For example, the cancer may be a recurrent or refractory HER2-positive cancer, preferably a recurrent or refractory cancer in which HER2 is genetically amplified and/or overexpressed. Example cancer types may include, but are not limited to, recurrent or refractory HER2-positive breast cancer or recurrent or refractory HER2-positive gastric cancer.

Subjects with a recurrent or refractory cancer whom have previously undergone at least one treatment regimen with one or more anticancer agents, preferably at least two treatment regimens with at least two different anticancer agents may be treated with Compound (1) or its pharmaceutically acceptable salt. In some cases, the recurrent or refractory cancer may have acquired resistance to, or intractability from, the prior treatment regimen(s). For example, a subject with a HER2-positive cancer treated previously with one or more anticancer agents, and that failed to respond to or relapsed from the prior treatment(s) with the anticancer agent(s), may develop resistance/intractability as a result of exposure of the cancer to the anticancer agent(s).

Resistance/intractability may manifest in the cancer in the form of EGFR and/or HER2 aberrations e.g., overexpression and/or mutations, or any other cancer driver alterations that result in loss-of-function of tumor suppressor genes/proteins or gain-of-function alterations in oncogenes/oncogene-encoded proteins.

Prior treatment regimen(s) may have been performed with a variety of anticancer agents, examples of such anticancer agents will be discussed hereinafter. A preferred embodiment of the present disclosure involves administering Compound (1) or its pharmaceutically acceptable salt to a subject with recurrent or refractory HER2-positive cancer that has previously undergone at least one treatment with, and optionally acquired resistance to or intractability from, one or more HER2 inhibitor(s) (i.e., HER2 inhibitors other than Compound (1)).

The HER2 inhibitor used in the prior treatment regimen(s) may be a HER2 tyrosine kinase inhibitor, examples of which include, but are not limited to, one or more of afatinib, lapatinib, neratinib, and tucatinib.

The HER2 inhibitor used in the prior treatment regimen(s) may be an anti-HER2 antibody or drug conjugate thereof, examples of which include, but are not limited to, one or more of trastuzumab, trastuzumab emtansine, pertuzumab, margetuximab, and trastuzumab deruxtecan (DS-8201a).

Before commencing treatment, determination may be made as to whether the subject has one or more aberrations in EGFR and/or HER2, as identified above. Thus, the methods may involve a pre-screening step to determine whether the subject has an aberration in EGFR and/or HER2 and is a good candidate for treatment. The aberration(s) may be determined from family history of cancers involving the aberration(s), by genotyping the subject or analyzing any biological sample from the subject including blood or tumor samples taken from the subject using assays such as those described hereinafter, or from historical records or previous testing performed on the subject. If the subject is determined to be EGFR-positive and/or HER2-positive, and to harbor one or more aberrations therein such as those described in the present disclosure, treatment with Compound (1) or its pharmaceutically acceptable salt is appropriate.

Predictive biomarkers which may be used to identify individuals who are likely to be responsive to treatment herein, include, but are not limited to, EGFR and/or HER2 overexpression/amplification or other gene alterations at DNA, RNA or protein level, for example, in breast cancer; EGFR/HER2 exon 20 insertions and other activating mutations, for example, in lung cancer (e.g., NSCLC); EGFRvIII mutation. EGFR amplification and/or O⁶-methylguanine-DNA methyltransferase (MGMT) promoter methylation, for example, in brain cancer (e.g., GBM). A companion diagnostic (CDx) test may be developed to analyze biological samples.

The presence of EGFR and/or HER2 aberrations (including aberrations that cause protein activation via aberrant ligand binding, e.g., NRG1 fusion-driven EGFR and/or HER2 aberrations as described above) may be determined, e.g., during subject pre-screening or from previous testing performed on the subject, or otherwise confirmed according to known assays, including cleared or approved in vitro diagnostic (IVD) assays or assays for this purpose. Examples of which include, but are not limited to, testing by next generation sequencing (NGS)-based gene panel, whole exome profiling, polymerase chain reaction (PCR), in situ hybridization (ISH), immunohistochemistry (IHC), flow cytometry, or other assays that can determine EGFR and/or HER2 aberrations on tumor tissues or circulating tumor DNA (ctDNA), RNA, protein, etc. For example, subjects who do not have archival tumor tissue samples can be biopsied and the fresh tumor biopsy can be analyzed for confirmation of existing aberrations.

The terms “administer”, “administering”, “administration”, and the like, refer to the methods that may be used to enable delivery of the active ingredient to the desired site of biological action. Routes or modes of administration are as set forth herein. These methods include, but are not limited to, oral routes, intraduodenal routes, parenteral injection (including intravenous, subcutaneous, intraperitoneal, intramuscular, intravascular, or infusion), topical/transdermal, and rectal/vaginal administration. Those of ordinary skill in the art are familiar with administration techniques that can be employed. Oral administration is preferred.

In the present disclosure, the term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”.

Regarding the administration schedule in the present application, “continuous” means administration every day without interruption during the treatment course. If the administration schedule follows an “intermittent” administration schedule, then one or more days of administration may be followed by one or more “rest days” or days of non-administration of drug within the course.

A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

The dosage amount and treatment duration are dependent on factors, such as bioavailability of a drug, administration mode, toxicity of a drug, gender, age, lifestyle, body weight, the use of other drugs and dietary supplements, the disease stage, tolerance and resistance of the body to the administered drug, etc., and then determined and adjusted accordingly. An appropriate dosage amount may differ from one individual to another. An appropriate dosage amount in any individual case may be determined using techniques, such as dose escalation.

The subject having at least one aberration in EGFR and/or HER2 can be treated with Compound (1) or its pharmaceutically acceptable salt at dose levels of from about 1 mg/day, from about 5 mg/day, from about 10 mg/day, from about 15 mg/day, from about 20 mg/day, from about 30 mg/day, from about 40 mg/day, from about 50 mg/day, from about 60 mg/day, from about 80 mg/day, from about 100 mg/day, from about 120 mg/day, and up to about 1,000 mg/day, up to about 800 mg/day, up to about 600 mg/day, up to about 500 mg/day, up to about 480 mg/day, up to about 450 mg/day, up to about 400 mg/day, up to about 350 mg/day, up to about 300 mg/day, up to about 250 mg/day, up to about 240 mg/day, up to about 200 mg/day, up to about 150 mg/day. The dosing level may be varied within the ranges such as from about 5 m_{g}/day to about 480 mg/day, from about 15 mg/day to about 240 mg/day, and from about 15 mg to about 120 mg/day. The administration dose level can be changed during an administration schedule, for example, the administration can begin with low dose for some time and then increased, or, the administration can begin with high dose for some time and then decreased.

The dosing can be continuous (daily; 7 days of administration in a week) or intermittent (alternating one or more dosing days with one or more non-dosing days, such as 4 days-on/3 days-off), for example, depending the pharmacokinetics and a particular patient's clearance/accumulation of the drug. The dosing schedule should be selected using sound medical judgement. Daily administration is preferred. The dosing can be performed every other day (QOD), once per day (QD), or more than once per day (b.i.d., t.i.d., etc.), with doses of about 15 to 480 mg/day being preferred.

The daily dose may be administered as a single dose or multiple individual divided doses. For example, three (3) tablets, each tablet containing 5 mg of Compound (1) or its pharmaceutically acceptable salt, may be administered to the subject once per day (QD) for a total dose of 15 mg/day. In another example, three (3) tablets, each tablet containing 20 mg of Compound (1) or its pharmaceutically acceptable salt, may be administered to the subject twice per day (b.i.d.) for a total dose of 120 mg/day.

The dosing whether continuous or intermittent is continued for a particular treatment cycle, typically at least a 28 day cycle, which can be repeated with or without a drug holiday. Longer or shorter cycles can also be used such as 7 days, 14 days, 18 days, 24 days, 28 days, 35 days, 42 days, or any range therebetween. The cycle may be repeated without a drug holiday or with a drug holiday depending upon the subject. A cycle of alternating and consecutive days can be used. For instance, a dosing schedule of sequential 7-day periods may be used where each period comprises alternating 4 days-on and 3 days-off. Here, this schedule would involve dosing on days 1, 3, 5, 7, 8, 10, 12, 14, 15, 17, 19, 21, and so on. Other schedules are possible depending upon the presence or absence of adverse events, response of the cancer to the treatment, patient convenience, and the like. An “adverse event” refers to any unfavorable or unintended illness or symptom thereof occurring in a patient to whom a drug has been administered. It does not matter whether there is a causal relationship with the drug or not.

The larger doses are usually given intermittently with doses up to about 480 mg usually given continuously (daily). Compound (1) may be dosed using an up-titration regimen, whereby a subject is started with a low dose for a certain period of time (e.g., 2 weeks) and then the dose is escalated. The dose may be up-titrated until either a target or maximum dose is reached or the subject experiences adverse events at which point the escalation is stopped and the drug dosing is reduced to a previous dose where the adverse event was not experienced or was not serious enough to require stoppage of the treatment. A subject that experiences an adverse event may also be managed with dosing interruptions (e.g., a drug holiday), if deemed appropriate. Typical dosing for the continuous regimen may be 15, 30, 60, 120, 240, or 480 mg/day but higher or lower doses may be used depending on the subject's response to the treatment and presence or absence of adverse events. If a dose is well-tolerated, the dose can be increased. The continuous administration may be continued for one cycle, e.g., 28 days, the cycle may then be repeated, as desired.

Such continuous or intermittent administration is applicable also to combination therapies where Compound (1) or its pharmaceutical acceptable salt is administered in combination with one or more other anticancer agents.

The treatment methods of the present disclosure may involve administration of Compound (1) or pharmaceutically acceptable salt thereof as a stand-alone therapy. The treatment may also involve administration as a post-operative auxiliary chemotherapy that is performed to prevent recurrence of tumors after surgically removing tumors, as well as pre-operative auxiliary chemotherapy prior to surgery to surgically remove tumors. In some cases, such as with breast cancer, surgery may include a lumpectomy, a mastectomy, a breast reconstruction, and the like. In some cases, such as with lung cancer, surgery may include pneumonectomy, lobectomy, wedge resection, sleeve resection, thoracoscopy, and the like. In some cases, such as with brain cancer, surgery may include craniotomy, ventriculoperitoneal shunt, endoscopic third ventriculostomy, surgery to put in a ventricular access catheter, and the like. The treatment may also include administration of Compound (1) or pharmaceutically acceptable salt thereof during or after radiation therapy or as an adjuvant therapy to prevent recurrence of the tumor in a patient where other treatments such as surgery have rendered the patient cancer-free.

Subjects may be treated herein whom have not previously undergone a treatment regimen with an anticancer agent(s), i.e., Compound (1) or its pharmaceutically acceptable salt are administered as first-line chemotherapy. Alternatively, as described heretofore, subjects may be treated whom have previously undergone a treatment regimen with one or more anticancer agents, i.e., Compound (1) or its pharmaceutically acceptable salt are administered as second-, third-, fourth-, etc. line therapy. Subjects may be treated whom have not previously undergone a chemotherapy regimen with an EGFR and/or HER2 inhibitor(s). Alternatively, subjects may be treated whom have been previously treated with an EGFR and/or HER2 inhibitor. Examples of HER2 inhibitors include, but are not limited to, those set forth herein. EGFR inhibitors may be categorized as an EGFR tyrosine kinase inhibitor or an anti-EGFR antibody, such as those set forth herein, examples of which include, but are not limited to, afatinib, gefitinib, erlotinib, osimertinib, lapatinib, neratinib, cetuximab, dacomitinib, panitumumab, vandetanib, and necitumumab. The subjects treated with Compound (1) or its pharmaceutically acceptable salt herein may, or may not, have been previously treated with anticancer agents besides EGFR and/or HER2 inhibitors, examples of which will be discussed hereinafter.

As described below, Compound (1) or its pharmaceutically acceptable salt may be specially formulated for administration in solid or liquid form, including those adapted for the following: (1) oral administration, for example, drenches (aqueous or non-aqueous solutions or suspensions), tablets or capsules, e.g., those targeted for buccal, sublingual, and systemic absorption, boluses, powders, granules, syrups, pastes for application to the tongue; (2) parenteral administration, for example, by subcutaneous, intramuscular, intravenous or epidural injection as, for example, a sterile solution or suspension, or sustained release formulation; (3) topical application/transdermal administration, for example, as a cream, ointment, or a controlled release patch or spray applied to the skin; (4) intravaginally or intrarectally, for example, as a pessary, cream or foam; or (5) nasally. In the case of Compound (1) or its pharmaceutically acceptable salt, an oral formulation is preferable.

Formulations can be prepared using a pharmaceutically acceptable carrier or the like by using known formulation methods. Pharmaceutically acceptable carriers are those materials, compositions, or vehicles, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide: (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations, such as cyclodextrins, liposomes, and micelle forming agents, e.g., bile acids, just to name a few.

Pharmaceutically acceptable carriers may be categorized as various general-purpose agents such as excipients, binders, disintegrating agents, lubricants, diluents, dissolution aids, suspending agents, swelling agents, isotonic agents, pH adjusters, buffers, stabilizers, colorants, flavoring agents, corrigents, and the like.

Examples of excipients include, but are not limited to, lactose, sucrose, D-mannitol, glucose, starch (corn starch), calcium carbonate, kaolin, microcrystalline cellulose, and silicic acid anhydride.

Examples of binders include, but are not limited to, water, ethanol, 1-propanol, 2-propanol, simple syrup, liquid glucose, liquid a-starch, liquid gelatin, D-mannitol, carboxymethyl cellulose, hydroxypropyl cellulose (e.g., low viscosity hydroxypropyl cellulose), hydroxypropyl methylcellulose (hypromellose), hydroxypropyl starch, methyl cellulose, ethyl cellulose, shellac, calcium phosphate, polyvinylpyrrolidone.

Examples of disintegrants include, but are not limited to, low-substituted hydroxypropyl cellulose, dry starch, partially pregelatinized starch, crystalline cellulose, carmellose sodium, carmellose calcium, D-mannitol, crospovidone, croscarmellose sodium, sodium alginate, agar powder, sodium hydrogen carbonate, calcium carbonate, sodium lauryl sulfate, stearic acid monoglyceride, and lactose.

Examples of lubricants include, but are not limited to, hydrogenated oil, sucrose fatty acid ester, sodium lauryl sulfate, stearic acid, purified talc, sodium stearate, magnesium stearate, borax, and polyethylene glycol.

Examples of colorants include, but are not limited to, edible yellow No. 5 dye, edible blue No. 2 dye, edible lake dye, iron sesquioxide, yellow sesquioxide, and titanium dioxide.

Examples of sweetening/flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, oil of spearmint, oil of sassafras, oil of clove, cinnamon, anethole, menthol, thymol, eugenol, eucalyptol, lemon, lime, and lemon-lime.

If desired, an enteric coating or a coating to increase the persistence of effects can be provided by methods desirable for oral preparations. Examples of such coating agents include hydroxypropyl methylcellulose, ethyl cellulose, hydroxymethyl cellulose, hydroxypropyl cellulose, polyethylene glycol, and Tween 80 (registered trademark).

Compound (1) or its pharmaceutically acceptable salt are preferably formulated in solid dosage form for oral administration, such as in the form of capsules, tablets, pills, dragees, powders, granules, troches, and the like, with preference given to film-coated tablets. Compound (1) or its pharmaceutically acceptable salt may be mixed with one or more pharmaceutically acceptable carriers such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants (e.g., fatty acid esters of sorbitan and polyalkolyated fatty acid esters of sorbitan such as Tween 80 (registered trademark); (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the formulations may also comprise buffering agents. Solid compositions of a similar type may also be employed as fillers in soft and hard shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methylcellulose), lubricant, inert diluent, preservative, disintegrant (for example, sodium starch glycolate or cross-linked sodium carboxymethyl cellulose), surface active or dispersing agent. Molded tablets may be made by molding in a suitable machine a mixture of the powdered compound moistened with an inert liquid diluent. The tablets, and other solid dosage forms may optionally be scored or prepared with coatings and shells, such as enteric coatings and other coatings well known in the pharmaceutical formulating art. One example coating formulation may include hypromellose, polyethylene glycol, titanium dioxide, and optionally a coloring agent. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria retaining filter, or by incorporating sterilizing agents in the form of sterile solid compositions which can be dissolved in sterile water, or some other sterile injectable medium immediately before use. These formulations may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above described excipients.

Compound (1) or its pharmaceutically acceptable salt may be formulated for parenteral administration, for intravenous, subcutaneous, intraperitoneal, intramuscular, intravascular, or infusion administration, by combining Compound (1) or its pharmaceutically acceptable salt with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and non-aqueous carriers which may be employed include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. These compositions may also contain adjuvants such as preservatives, wetting agents, emulsifying agents, dispersing agents, pH regulators, stabilizers, local anesthetics, etc. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Compound (1) or its pharmaceutically acceptable salt can be combined with one or more anticancer agents, such as those described in Japanese Patent Application No. 2020-121733, the disclosure of which is incorporated herein by reference. Examples of anticancer agents include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anticancer agents have been categorized within a specific group(s) or subgroup(s) herein, many of these agents can also be listed within one or more other group(s) or subgroup(s), as would be presently understood in the art. The anticancer agent is not particularly limited, and examples thereof include, but are not limited to, a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAG1 agent, an anti-OX40 agent, a GITR agonist, a CAR-T cell, a BiTE, a signal transduction inhibitor, a growth factor inhibitor, a tyrosine kinase inhibitor, an EGFR inhibitor, a HER2 inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor a HIF-2a inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a BRAF-inhibitor, a RAS inhibitor, a gene expression modulator, an autophagy inhibitor, an apoptosis inducer, an antiproliferative agent, and a glycolysis inhibitor.

Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine: vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; cyclophosphamide; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), tegafur/gimeracil/oteracil potassium, tegafur/uracil, trifluridine, trifluridine/tipiracil hydrochloride, 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur: and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epinibicin, idarubicin, menogaril, nogalamycin, pirarubicin, and valrubicin; 6-diazo-5-oxo-L-norleucine: aclacinomysins; actinomycin; authramycin; azaserine; bleomycins; cactinomycin; calicheamicin; carabicin; carminomycin; carzinophilin; chromomycins; dactinomycin; detorubicin; esonibicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles. EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

Non-limiting examples of immunotherapeutic agents (i.e., immunotherapy) include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, veltuzumab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilumumab and tremelimumab; anti-LAG1 agents; and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, HER2 inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, RAF inhibitor, MEK inhibitors, ERK inhibitors, PI3K inhibitors, AKT inhibitors, MCL-1 inhibitors, BCL-2 inhibitors, SHP2 inhibitors. BRAF-inhibitors, RAS inhibitor, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR and/or HER2 inhibitory agents (i.e., other than Compound (1) or its salt).

Non-limiting examples of EGFR inhibitors include small molecule antagonists of EGFR such as afatinib, brigatinib, erlotinib, gefitinib, lapatinib, neratinib, dacomitinib, vandetanib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1 1311-1318; Huang, S. M., et al., 1999, Cancer Res. 15:59(8): 1935-40; and Yang, X., et al., 1999, Cancer Res. 59: 1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of HER2 inhibitors include HER2 tyrosine kinase inhibitors such as afatinib, lapatinib, neratinib, and tucatinib; and anti-HER2 antibodies or drug conjugates thereof such as trastuzumab, trastuzumab emtansine (T-DM1), pertuzumab, margetuximab, trastuzumab deruxtecan (DS-8201a), and trastuzumab duocarmazine.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

Non-limiting examples of anti-angiogenic agents (or angiogenesis inhibitors) include, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib: HIF-1α inhibitors such as PX 478; HIF-2α inhibitors such as belzutifan and the HIF-2a inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™.

Other anti-angiogenic agents may include, but are not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and MedImmune, US), MAb VEGF (Xenova, UK), marimastat, maspin (Sosei, Japan), metastatin, motuporamine C, M-PGA, ombrabulin, OXI4503. PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

The anticancer agent(s) that may be combined with Compound (1) may also be an active agent that disrupts or inhibits RAS-RAF-ERK or PI3K-AKT-TOR signaling pathways or is a PD-1 and/or PD-L1 antagonist. Examples of which include, but are not limited to, a RAF inhibitor, an EGFR inhibitor, a MEK inhibitor, an ERK inhibitor, a PI3K inhibitor, a AKT inhibitor, a TOR inhibitor, an MCL-1 inhibitor, a BCL-2 inhibitor, a SHP2 inhibitor, a proteasome inhibitor, or an immune therapy, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and ASTX029.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; taselisib (GDC-0032); and ZSTK474.

Non-limiting examples of AKT inhibitors include Akt-1-1 (inhibits Akt1) (Barnett et al. (2005) Biochem. J., 385 (Pt. 2), 399-408); Akt-1-1,2 (Barnett et al. (2005) Biochem. J 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134 (12 Suppl), 3493S-3498S); perifosine, Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9): imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; 8-[4-(1-aminocyclobutyl)phenyl]-9-phenyl-1,2,4-triazolo[3,4-f][1,6]naphthyridin-3(2H)-one (MK2206) and pharmaceutically acceptable salts thereof; AZD5363; trans-3-amino-1-methyl-3-(4-(3-phenyl-5H-imidazo[1,2-c]pyrido[3,4-e][1,3]oxazin-2-yl)phenyl)cyclobutanol (TAS117) and pharmaceutically acceptable salts thereof; and pataserib.

Non-limiting examples of TOR inhibitors include deforolimus; ATP-competitive TORC1/TORC2 inhibitors, including PI-103, PP242, PP3, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin; 40-epi-(tetrazolyl)-rapamycin (also called ABT578); AZD8055; 32-deoxorapamycin: 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include JAB-3068, RMC-4630, TNO155, SHP-099, RMC-4550, and SHP2 inhibitors described in WO 2019/167000, WO 2020/022323 and WO2021/033153.

Non-limiting examples of RAS inhibitors include AMG510, MRTX849, LY3499446, JNJ-74699157 (ARS-3248), ARS-1620, ARS-853, RM-007, and RM-008.

Additional non-limiting examples of anticancer agents that may be suitable for use include, but are not limited to, 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflornithine, EL532 (Elan), elfomithine, elsamitrucin, eniluracil, etanidazole, exisulind, ferriginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galdernma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

As used in the present disclosure, the term “combination,” “combined.” or a variation thereof is intended to define a therapy involving the use of two or more compound/drug combinations. The term can refer to compounds/drugs that are administered as part of the same overall dosage schedule. The respective dosages of two or more compounds/drugs can be different. The combination therapy is intended to embrace administration of these compounds/drugs in a sequential manner, that is, wherein each compound/drug is administered at a different time, as well as administration of these compounds/drugs, or at least two of the compounds/drugs, in a substantially simultaneous manner. Substantially simultaneous administration can be accomplished, for example, by administering to the subject a single dosage form having a fixed ratio of each compound/drug or in multiple, single dosage forms for each of the compounds/drugs. Sequential or substantially simultaneous administration of each compound/drug can be effected by any appropriate route including, but not limited to, oral routes, intravenous routes, intramuscular routes, and direct absorption through mucous membrane tissues (e.g., buccal). The compounds/drugs can be administered by the same route or by different routes. For example, a first compound/drug of the combination selected may be administered by intravenous injection while the other compound/drug of the combination may be administered orally. Alternatively, for example, all compounds/drugs may be administered orally or all compounds/drugs may be administered by intravenous injection.

Combination therapy also can embrace the administration of the compounds/drugs as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of compound/drug and non-drug treatment is achieved. For example, in appropriate cases, the beneficial effect is still achieved when the non-drug treatment is temporally removed from the administration of the compound/drug, perhaps by days or even weeks.

Techniques for administering radiation therapy are known in the art, and these techniques can be used in the combination therapy described herein. The administration of Compound (1) in this combination therapy can be determined as described herein. Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

### EXAMPLES

### Synthesis of Compound (1)

### <Step I> tert-Butyl (2S,4R)-4-(4-amino-5-iodo-7H-pyrrolo[2,3-d]pyrimidin-7-yl)-2-methylpyrrolidine-1-carboxylate

tert-Butyl (2S,4S)-4-hydroxy-2-methylpyrrolidine-1-carboxylate (19.0 g) and 4-chloro-5-iodo-7H-pyrrolo[2,3-d]pyrimidine (13.1 g) were dissolved in THF (190 mL), and the obtained solution was then cooled to 0° C. Thereafter, triphenylphosphine (37.2 g) and diisopropyl azodicarboxylate (28.1 mL) were added to the reaction solution, and the temperature of the mixture was then increased to room temperature, followed by stirring for 1 hour. Thereafter, the reaction mixture was concentrated under reduced pressure, and the obtained residue was then purified by silica gel chromatography (hexane: ethyl acetate) to obtain the corresponding coupling body. The obtained compound was used in the subsequent reaction without being further purified.

The obtained coupling body, THF (114 mL) and ammonia water (114 mL) were added into a pressure resistant tube, and the obtained mixture was then stirred at 100° C. for 14 hours. Thereafter, the reaction mixture was cooled to room temperature, and was then poured into water (285 mL). The thus obtained mixture was stirred at room temperature for 5 hours. Thereafter, the precipitated solid was collected by filtration, was then washed with water, and was then dried to obtain a product of interest (34.5 g).

¹HNMR (CDCl₃) δ: 8.27 (s, 1H) 7.15 (s, 1H) 5.55-5.73 (m, 2H) 5.12-5.25 (m, 1H) 3.86-4.18 (m, 2H) 3.43-3.57 (m, 1H) 2.59-2.69 (m, 1H) 1.92-2.03 (m, 1H) 1.48 (s, 9H) 1.30-1.40 (m, 3H).

ESI-MS m/z 444 (MH+).

### <Step 2> 4-Amino-7-((3R,5S)-1-(tert-butoxycarbonyl)-5-methylpyrrolidin-3-yl)-7H-pyrrolo[2,3-d]pyrimidine-5-carboxylic acid

The compound of Step 1 (28.0 g), 10% palladium carbon catalyst (720 mg), NMP (84 mL), methanol (26 mL), and triethylamine (17.6 mL) were added into a pressure resistant tube, followed by carbon monoxide substitution, and the obtained mixture was stirred at 100° C. for 2 hours. Thereafter, the reaction mixture was cooled to room temperature, a 2 M sodium hydroxide aqueous solution (79 mL) was then added thereto, and the obtained mixture was then stirred at 80° C. for 2 hours. Thereafter, the reaction mixture was cooled to room temperature, was then filtrated through Celite, and was then washed with methanol. Subsequently, methanol in the filtrate was concentrated under reduced pressure. Water was further added, and the water layer was then washed with tert-butyl methyl ether. A 1 M potassium hydrogen sulfate aqueous solution was added to the water layer to adjust the pH to approximately 3. The precipitated solid was collected by filtration, was then washed with water, and was then dried to obtain a product of interest (23.4 g).

¹HNMR (400 MHz, DMSO-d6) δ: 8.14 (s, 1H) 8.08 (s, 1H) 5.16-4.93 (m, 1H) 4.07-3.79 (m, 2H) 3.61-3.45 (m, 1H) 2.53 (m, 1H) 2.33-2.02 (m, 1H) 1.42 (s, 9H) 1.29 (d, J=6.1 Hz, 3H).

ESI-MS m/z 362 (MH+).

### <Step 3> tert-Butyl(2S,4R)-4-(4-amino-6-bromo-5-(((R)-1-phenylethyl)carbamoyl)-7H-pyrrolo[2,3-d]pyrimidin-7-yl)-2-methylpyrrolidine-1-carboxylate

The compound of Step 2 (1.00 g), (R)-(+)-1-phenylethylamine (0.503 g), diisopropylethylamine (1.79 g), and N,N-dimethylformamide (10 mL) were added, and subsequently, HATU (1.58 g) was added. The obtained mixture was stirred at room temperature overnight. Thereafter, to the reaction mixture, ethyl acetate and a saturated sodium hydrogen carbonate aqueous solution were added, and the obtained mixture was then extracted with ethyl acetate. The gathered organic layer was washed with water, and then with saturated saline. The resultant was dried over anhydrous sodium sulfate, and was then concentrated under reduced pressure. The obtained residue was purified by silica gel chromatography (hexane: acetone) to obtain an amide form (1.53 g). The obtained compound was used in the subsequent reaction without being further purified.

To the amide form (1.53 g), chloroform (15 mL) was added, and the obtained mixture was then cooled to 0° C. Thereafter, N-bromosuccinimide (0.88 g) was added to the reaction mixture, and the obtained mixture was then stirred at 0° C. for 1 hour. Thereafter, the reaction mixture was concentrated under reduced pressure, and the obtained residue was purified by silica gel chromatography (hexane: ethyl acetate) to obtain a product of interest (1.39 g).

¹HNMR (CDCl₃) δ: 8.21 (s, 1H) 7.42-7.28 (m, 5H) 6.97 (d, J=7.3 Hz, 1H) 5.36-5.29 (m, 1H) 5.20-5.07 (m, 1H) 4.30 (t, J=10.3 Hz, 1H) 4.04-3.72 (m, 2H) 3.00-2.86 (m, 1H) 2.38 (dt, J=14.3, 6.0 Hz, 1H) 1.63 (d, J=7.0 Hz, 3H) 1.53-1.43 (m, 12H).

ESI-MS m/z 543,545 (MH+).

### <Step 4> 7-((3R,5S)-1-acryloyl-5-methylpyrrolidin-3-yl)-4-amino-6-bromo-N—((R)-1-phenylethyl)-7H-pyrrolo[2,3-d]pyrimidine-5-carboxamide

To the compound of Step 3 (600 mg), chloroform (3 mL) was added, and the obtained mixture was then cooled to 0° C. Thereafter, trifluoroacetic acid (4.44 g) was added to the reaction mixture, and the thus obtained mixture was then stirred at room temperature for 1 hour. Thereafter, the reaction mixture was concentrated under reduced pressure, and acetonitrile (5 mL) was then added to the residue. The obtained mixture was concentrated under reduced pressure again to obtain an amine form. The obtained compound was used in the subsequent reaction without being further purified.

To the obtained amine form, acetonitrile (3 mL) was added, and the obtained mixture was then cooled to 0° C. Thereafter, acryloyl chloride (99.9 mg) and diisopropylethylamine (713 mg) were added, and the obtained mixture was then stirred at 0° C. for 1 hour. Thereafter, the reaction mixture was concentrated under reduced pressure, and the obtained residue was purified by silica gel chromatography (ethyl acetate methanol) to obtain a product of interest (281 mg).

¹HNMR (CDCl₃) δ: 8.20 (d, J=7.3 Hz, 1H) 7.42-7.36 (m, 4H) 7.32-7.28 (m, 1H) 7.00-6.94 (m, 1H) 6.57-6.33 (m, 2H) 5.76-5.66 (m, 1H) 5.36-5.29 (m, 1H) 5.14-5.08 (m, 1H) 4.71 (t, J=9.9 Hz, 0.7H) 4.42-4.23 (m, 1.6H) 3.83 (t, J=8.6 Hz, 0.7H) 3.03-2.92 (m, 1H) 2.60-2.57 (m, 0.3H) 2.44-2.40 (m, 0.7H) 1.64 (d, J=6.6 Hz, 3H) 1.56 (dd, J=11.7, 6.2 Hz, 3H).

ESI-MS m/z 497,499 (MH+).

### <Step 5> 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide (Compound (1))

The compound of Step 4 (65 mg), dichlorobis(triphenylphosphine)palladium (9.2 mg), copper(I) iodide (5.0 mg), cyclopropylacetylene (13.0 mg), triethylamine (39.7 mg), and N,N-dimethylformamide (1.3 mL) were added, and the inside of the reaction system was then substituted with nitrogen. After that, the mixture was stirred at 70° C. for 2.5 hours. Thereafter, to the reaction mixture, ethyl acetate and a saturated ammonium chloride aqueous solution were added, and the obtained mixture was then extracted with ethyl acetate. The gathered organic layer was washed with water, and then with saturated saline. The resultant was dried over anhydrous sodium sulfate, and was then concentrated under reduced pressure. The obtained residue was purified by silica gel chromatography (chloroform:methanol) to obtain a product of interest (50 mg).

¹HNMR (CDCl₃) δ: 8.22 (d, J=5.1 Hz, 1H) 7.82 (d, J=7.3 Hz, 1H) 7.43-7.35 (m, 4H) 7.30 (t, J=6.8 Hz, 1H) 6.58-6.34 (m, 2H) 5.77-5.66 (m, 1H) 5.35-5.20 (m, 2H) 4.54 (t, J=10.1 Hz, 0.7H) 4.35-4.25 (m, 1.6H) 3.88 (t, J=8.8 Hz, 0.7H) 2.90-2.78 (m, 1H) 2.65-2.56 (m, 0.3H) 2.49-2.40 (m, 0.7H) 1.63 (d, J=7.0 Hz, 3H) 1.56-1.45 (m, 4H) 1.03-0.91 (m, 2H) 0.84-0.69 (m, 2H).

ESI-MS m/z 483 (MH+).

### Production of Free-Form Type I Crystal of Compound (1)

A crude product (100 mg) of Compound (1) was suspended in ethanol (500 μL) and stirred at 50° C. overnight. The solution was cooled to 25° C. and the obtained suspension was filtrated to obtain a free-form type I crystal of Compound (1) (31 mg).

Production of Type II Crystal of Compound (1) with 1 Eq of Fumaric Acid (Monofumarate Salt)

Type II crystals of Compound (1) with fumaric acid were produced by the two methods described below in Production Method 1 or Production Method 2.

<Production method 1> Fumaric acid (3 equivalents) was added to 30 mg of a free-form type I crystal of Compound (1), 0.3 mL of tert-butanol was added, the suspension was stirred at 25° C. for about 124 hours, and then the solid was collected by filtration, recovered, and dried to obtain a crystal of interest.

<Production method 2> Fumaric acid (1.06 g) and 22 mL of acetone were added to 2.20 g of a free-form type I crystal of Compound (I), the suspension was stirred at 50° C. for about 62 hours, naturally cooled for 30 minutes, and then the solid was collected by filtration and recovered. This was washed with 2-propanol, and then the solid was collected by filtration, recovered, and dried to obtain 2.27 g of a crystal of interest.

### Formulation

Compound (1) was administered in all nonclinical studies in the form of a monofumarate salt (C₂₈H₃₀N₆O₂·C₄H₄O₄), unless otherwise specified. All references to Compound (1) throughout the examples below designate this salt form. The doses are presented as free base equivalents (i.e., equivalents of Compound (1)). When formulated in aqueous oral suspension dosage form, the active drug substance was suspended in 0.5% weight per volume (w/v) hydroxypropyl methylcellulose (HPMC) as vehicle. When formulated in film-coated tablet dosage form, the active drug substance was combined with the following inactive ingredients: lactose monohydrate, microcrystalline cellulose, croscarmellose sodium, talc, light anhydrous silicic acid, magnesium stearate, hypromellose, polyethylene glycol, and titanium dioxide.

### Inhibitory Effects of Compound (1) on HER2 Enzyme Activity

To investigate the inhibitory effect of Compound (1) on HER2 enzyme activity, the mean±standard deviation (SD) of half-maximal inhibitory concentration (IC₅₀) values of Compound (1) against human recombinant enzymes of HER2 and its mutants were determined by in vitro kinase assays (Table 1). These results showed that Compound (1) inhibits the kinase activity of HER2 and its mutants, with particularly high potency against HER2 exon 20 insertion mutants.

TABLE 1
IC₅₀ values of Compound (1) against
HER2 and its mutants in enzyme assay

| Kinase | IC₅₀ (Mean ± SD, nmol/L Compound (1)) |
|---|---|
| HER2 | 5.6 ± 0.3 |
| HER2 V777L | 2.1 ± 0.8 |
| HER2 A775_G776insYVMA | 1.0 ± 0.2 |

### Inhibitory Effects of Compound (1) on EGFR Enzyme Activity

To investigate the inhibitory effect of Compound (1) on EGFR enzyme activity, the mean±standard deviation (SD) of half-maximal inhibitory concentration (IC₅₀) values of Compound (1) against human recombinant enzymes of EGFR and its mutants were determined by in vitro kinase assays (Table 2). These results showed that Compound (1) inhibits the kinase activity of EGFR and its mutants, notably including EGFR exon 20 insertion mutants.

TABLE 2
IC₅₀ values of Compound (1) against
EGFR and its mutants in enzyme assay

| | IC₅₀ (Mean ± SD, |
|---|---|
| Kinase | nmol/L Compound (1)) |
| EGFR | 0.47 ± 0.04 |
| EGFR DelE746_A750 | 0.25 ± 0.03 |
| EGFR DelE746_A750/T790M | 6.8 ± 0.3 |
| EGFR L858R | 0.36 ± 0.03 |
| EGFR T790M/L858R | 6.6 ± 0.2 |
| EGFR D770_N771insNPG | 0.55 ± 0.06 |

### Enzyme Inhibition Selectivity of Compound (1)

To investigate further kinase selectivity of Compound (1), a panel enzyme assay of 257 kinases for Compound (1) was conducted. Compound (1) showed an inhibition rate of higher than 50% (≥50% inhibition of activity) for only 14 of the 257 kinases tested at a concentration of 1000 nmol/L (Table 3). Compound (1) showed a weak (<50% inhibition of activity) inhibition or no inhibitory effect for the other 243 kinases.

TABLE 3
Percent inhibition values of Compound (1) for 14 kinases with
values ≥50% among 257 kinases including ErbB family proteins

| Kinase | % Inhibition by Compound (1) at 1000 nmol/L |
|---|---|
| HER2 | 100.1 |
| JAK3 | 99.2 |
| BMX | 98.9 |
| HER4 | 98.4 |
| EGFR | 98.3 |
| TXK | 89.4 |
| DDR1 | 86.0 |
| TEC | 81.7 |
| FLT4 | 80.4 |
| BTK | 80.0 |
| EPHA6 | 72.9 |
| KDR | 67.2 |
| BLK | 64.7 |
| ITK | 56.4 |

### Evaluation of Inhibitory Effects of Compound (1) on Feedback Activation of HER3 in SK-BR-3 Human Breast Adenocarcinomna Cell Lines

The effect of Compound (1) on feedback activation of HER3, its downstream targets (ERK and AKT), and apoptosis markers (BIM and cleaved PARP) in SK-BR-3 cells was investigated. In addition, the same information for lapatinib and tucatinib (a reversible inhibitor of HER2) was obtained.

Compound (1) showed sustained inhibition of phosphorylation of HER2, HER3, AKT, and ERK at concentrations starting as low as 10 nmol/L for 48 hours.

BIM and cleaved PARP were markedly increased after treatment of SK-BR-3 cells with Compound (1) for 48 hours at concentrations from 10 to 100 nmol/L.

Treatment of SK-BR-3 cells with lapatinib and tucatinib for 3 hours at concentrations ranging from 30 to 300 nmol/L also inhibited the phosphorylation of HER2, HER3, and their downstream targets. However, at 48 hours the phosphorylation of HER2, HER3, and their downstream targets (AKT and ERK) was increased again. Also, BIM and cleaved PARP were increased after treatment of SK-BR-3 cells with lapatinib and tucatinib at 48 hours at concentrations from 30 to 300 nmol/L.

Evaluation of the Growth Inhibitory Effect of Compound (1) in Human Cancer Cell Lines with ErbB1/2 Amplification or Mutations

To investigate the inhibitory effect of Compound (1) on the proliferation of human cancer cell lines with ErbB1/2 amplification or mutations, the 50% growth inhibitory concentration (GI₅₀) values were determined for each cell line.

Among the 9 cell lines tested, HER2 gene amplification has previously been reported in BT-474 cells (breast cancer) and NCI-N87 cells (gastric cancer), while HER2 gene mutation (DelG776insVC) has been reported in NCI-H1781 cells (lung cancer).

Compound (1) inhibited the proliferation of these 3 cell lines, with GI₅₀ values of 2.05±0.48 nmol/L, 0.891±0.312 nmol/L, and 5.85±2.83 nmol/L, respectively. In addition, Compound (1) inhibited the proliferation of MCF10A_HER2/insYVMA cells, a human mammary epithelial cell line engineered to express the HER2 (A775_G776insYVMA), with a GI₅₀ value of 23.3±1.4 nmol/L.

Wild-type EGFR gene amplification without HER2 amplification has been observed in A-431 cells. HCC827 cells (lung cancer) have been reported as harboring an EGFR activating mutation (DelE746_A750). NCI-H1975 cells (lung cancer) have been reported as having activating and acquired EGFR resistance mutations (L858R and T790M). The GI₅₀ values for these cell lines after exposure to Compound (1) were 8.64±1.07 nmol/L (A-431), 2.13±0.61 nmol/L (HCC827), and 37.5±20.4 nmol/L (NCI-H1975).

Proliferation of NCI-H1975 EGFR D770_N771insSVD cells (lung cancer) expressing exon 20 insertion mutations (activating and intrinsic resistance mutations) has also been shown to be inhibited by Compound (1) with a GI₅₀ value of 8.71±4.56 nmol/L. In contrast, the G1₅₀ value of NCI-H460 (lung cancer) cells expressing KRAS Q61H mutation without ErbB1/2 aberration following Compound (1) exposure was >1000 nmol/L.

Thus, Compound (1) has demonstrated a selective and potent inhibitory effect on the proliferation of human cancer cells expressing ErbB1/2 amplification or various ErbB1/2 mutations including exon 20 insertions.

### Evaluation of the Antitumor Effects of Compound (1) in Nude Mice Bearing NCI-N87 Human Gastric Carcinoma Xenografts

The antitumor effect of Compound (1) was evaluated in nude mice bearing HER2-overexpressing NCI-N87 human gastric carcinoma xenografts. Compound (1) dosing suspensions using 0.5% w/v hydroxypropyl methylcellulose (HPMC) as vehicle were prepared at 4 dose levels (3.1, 6.3, 12.5, and 25.0 mg/kg/day) and administered orally once daily for 14 days. In addition, the antitumor effect of poziotinib, which is a pan-ErbB inhibitor and currently under clinical development for the treatment of cancer with epidermal growth factor receptor containing HER2 exon 20 insertion mutations, was also evaluated using this model. Poziotinib was administered orally once daily for 14 days at 2 dose levels (0.5 or 1.0 mg/kg/day). The control group received 0.5% w/v HPMC once daily for 14 days.

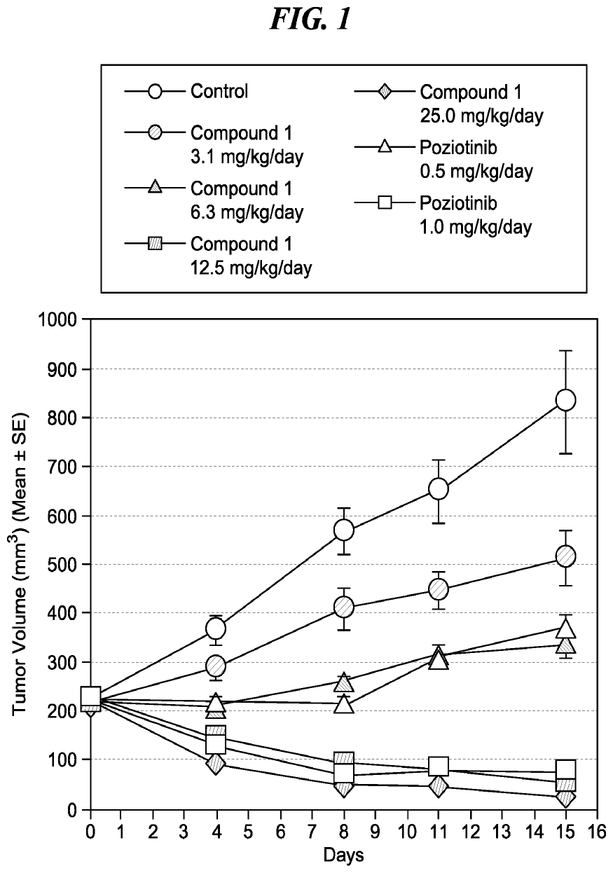

At the evaluation on Day 15, all groups treated with the test compounds showed significant tumor growth inhibition compared with the control group (FIG. 1). The treated group/control group (T/C) ratios of the mean tumor volumes (TVs) were 61.5%, 40.7%, 7.3%, and 3.4% in the 3.1, 6.3, 12.5, and 25.0 mg/kg/day Compound (1) groups, respectively, and 44.2% and 8.8% in the 0.5 and 1.0 mg/kg/day poziotinib groups, respectively.

No mouse had more than a 20% loss in body weight from Day 0 during the study period. These findings indicated that Compound (1) at doses of 3.1 to 25.0 mg/kg/day showed antitumor activity in a dose-dependent manner, and caused no severe body weight loss in nude mice bearing NCI-N87 human gastric carcinoma xenografts.

Target protein evaluation. To examine pharmacodynamics at the efficacious dosage of Compound (1), the levels of phospho-HER2, phospho-HER3, phospho-ERK, and phospho-AKT were investigated in tumor tissue in a nude mouse model with subcutaneous implantation of NCI-N87 human gastric carcinoma.

In the 3.1-, 6.3-, 12.5-, and 25.0-mg/kg groups, tumor samples were collected 1 hour after Compound (1) administration. In the 12.5- and 25.0-mg/kg groups, additional tumor samples were collected 2, 4, 6, 12, and 24 hours after Compound (1) administration. The control group received 0.5% weight per volume (w/v) hydroxypropyl methylcellulose (HPMC) solution after single oral administration and tumor samples were collected immediately after administration.

At 1 hour after administration of Compound (1), compared with the control group, treatment with Compound (1) (from 3.1 to 25.0 mg/kg) decreased the phosphorylation of HER2 and HER3 without altering the total levels of these 2 proteins in a dose-dependent manner. Additionally, it was observed that the phosphorylation of ERK and AKT (the substrate proteins or target proteins) was also downregulated at the same point.

The downregulation of HER2 and HER3 phosphorylation in NCI-N87 tumor tissues continued from 1 to 4 hours after Compound (1) administration at a dose of 12.5 mg/kg. At a dose of 25.0 mg/kg, downregulation of HER2 and HER3 phosphorylation in tumor tissues continued from 1 to 12 hours. Phosphorylation of AKT and ERK, including ERK1 and ERK2 (which are downstream targets of HER2 and HER3) was also downregulated at 1 hour; these downregulations continued through 12 hours after Compound (1) administration. In addition, expression of cleaved PARP (a marker of apoptosis) and BIM (a key effector of apoptosis) were increased by Compound (1) administration at both doses.

Since Compound (1) decreased the phosphorylation of target proteins and their substrates and increased the level of apoptosis-related factors, Compound (1) is believed to exert an antitumor effect via inactivation of the HER2 signaling pathway.

### Evaluation of the Antitumor Effects of Compound (1) in Nude Mice Bearing MCF10A_HER2/insYVMA_v Human Mammary Epithelial Xenografts

The antitumor effect of Compound (1) was evaluated in a nude mouse xenograft model of MCF10A_HER2/insYVMA_v. MCF10A_HER2/insYVMA_v was established as stably growing cells in vivo derived from MCF10A HER2/insYVMA (a human mammary epithelial cell line engineered to express the HER2 exon 20 insertion mutation A775_G776insYVMA) xenografts subcutaneously transplanted into nude mice. Compound (1) suspensions (using 0.5% w/v HPMC as vehicle) were administered orally once daily to mice at doses of 7.8, 12.5, or 20.0 mg/kg/day for 14 days. The control group received 0.5% weight per volume (w/v) hydroxypropyl methylcellulose (HPMC) solution orally once daily for 14 days.

In addition, the antitumor effect of poziotinib (an irreversible inhibitor of EGFR, HER2, and HER4) was also evaluated using this model.

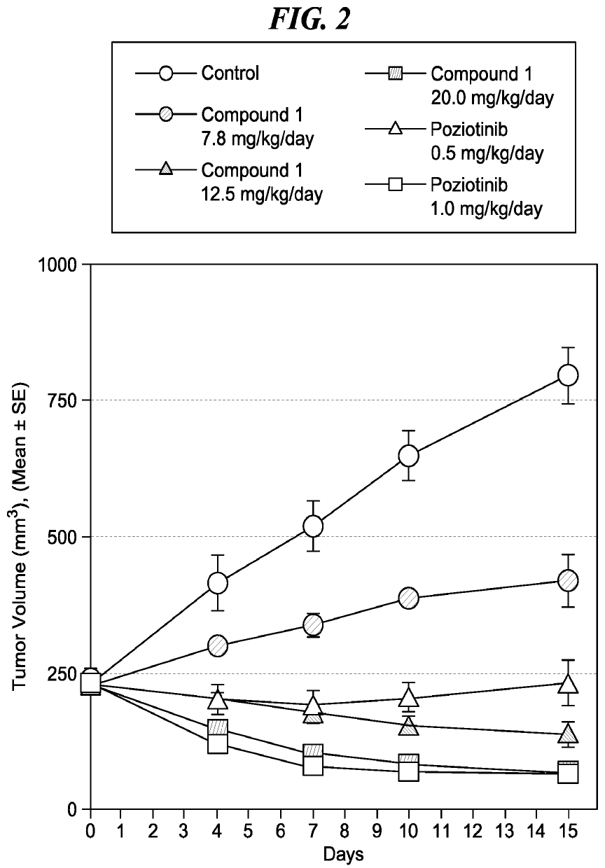

The antitumor effects during treatment with Compound (1) and poziotinib are presented in FIG. 2.

In the Compound (1) treatment groups, the T/C ratios (%) of the mean tumor volume (TV) values on Day 15 in the groups receiving 7.8, 12.5, and 20.0 mg/kg/day were 52.8%, 17.4%, and 8.7%, respectively. The mean log₁₀ TVs on Day 15 in all Compound (1) treatment groups were significantly lower than that in the control group (P=0.0016, P<0.0001 and P<0.0001, respectively).

In the poziotinib treatment groups, the T/C ratios (%) of the mean TVs on Day 15 in the groups receiving 0.5 and 1.0 mg/kg/day were 29.0% and 8.3%, respectively. The mean log₁₀ TVs on Day 15 in both poziotinib treatment groups were significantly lower than that in the control group (P<0.0001 for each).

Since none of the mice died and no abnormal clinical signs or a body weight loss ≥20% were observed, the toxicity was judged to be tolerable in all the test article treatment groups. Compound (1) thus showed antitumor effects with an acceptable toxicity in a nude mouse xenograft model of the human mammary epithelial cell line harboring HER2 exon 20 insertion mutation, MCF10A_HER2/insYVMA_v.

Evaluation of the Antitumor Effects of Compound (1) in Nude Mice Bearing Intracranial NCI-N87-Luc Human Gastric Carcinoma Xenografts

The intracranial antitumor effect of Compound (1) was evaluated using Compound (1) in nude mice intracranially implanted with NCI-N87-luc cells, which were engineered to stably express the luciferase gene using the HER2-overexpressing NCI-N87 human gastric carcinoma cell line. Compound (1) dosing suspensions using 0.5% w/v HPMC as vehicle were prepared with Compound (1) at 3 dose levels (16.0, 20.0, or 25.0 mg/kg/day) and administered PO QD for 21 days. The control group received 0.5% weight per volume (w/v) hydroxypropyl methylcellulose (HPMC) solution orally once daily for 21 days. The antitumor effect of poziotinib was also evaluated using this model. Poziotinib was administered PO QD for 21 days at 2 dose levels (0.5 or 1.0 mg/kg/day).

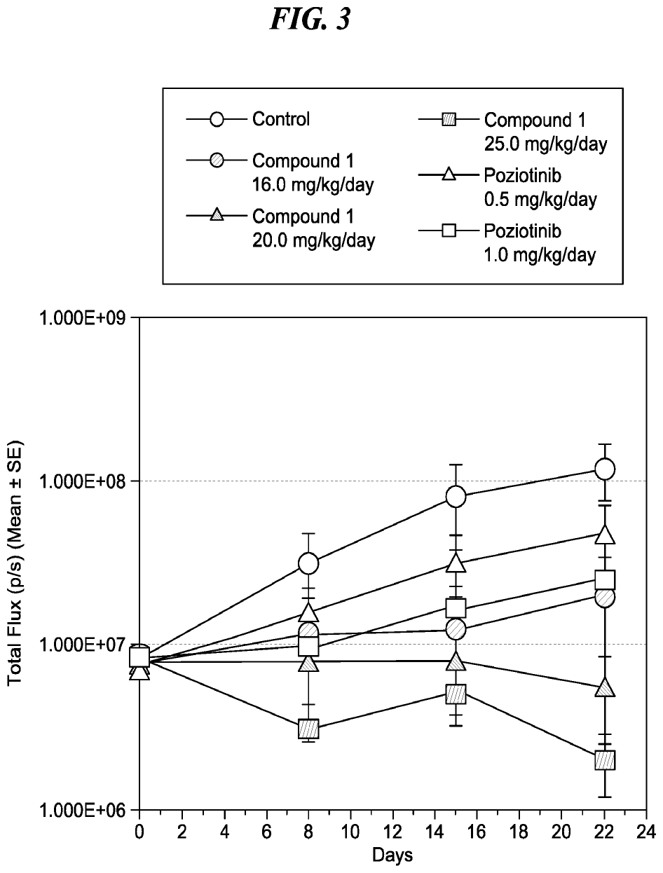

At the evaluation on Day 22, all groups treated with Compound (1) showed significant tumor growth inhibition compared with the control group (P<0.05, Dunnett's test) (FIG. 3). In the poziotinib-treated groups, the mean logarithmically transformed total flux values on Day 22 were significantly smaller in the group receiving 1.0 mg/kg/day as compared to the control group (P<0.05), but not in the group receiving 0.5 mg/kg/day (P=0.57) (FIG. 3).

The T/C ratios of the mean total flux in the groups treated with Compound (1) were 16.9%. 4.7%, and 1.7% at 16.0, 20.0, and 25.0 mg/kg/day, respectively; and the T/C ratios in the poziotinib-treated groups were 39.0% and 21.1% at 0.5 and 1.0 mg/kg/day, respectively. No mice had >20% loss in body weight from Day 0 during the treatment period. For the groups treated with Compound (1), mean body weight changes (BWCs) on Day 22 were 1.4%, −0.4%, and −2.8% at 16.0, 20.0, and 25.0 mg/kg/day, respectively. For the groups treated with poziotinib, mean BWCs were 2.5% and −0.3%, respectively.

These findings indicated that Compound (1) showed intracranial antitumor activity in a dose-dependent manner without severe body weight loss in nude mice implanted with NCI-N87-luc human gastric carcinoma in the brain parenchyma; with superior intracranial antitumor effects compared to poziotinib.

### Nonclinical Pharmacokinetics

Compound (1) was orally administered. The concentrations of Compound (1) were quantified using LC-MS/MS for the PK studies. ¹³C-labeled Compound (1) was used as an internal standard.

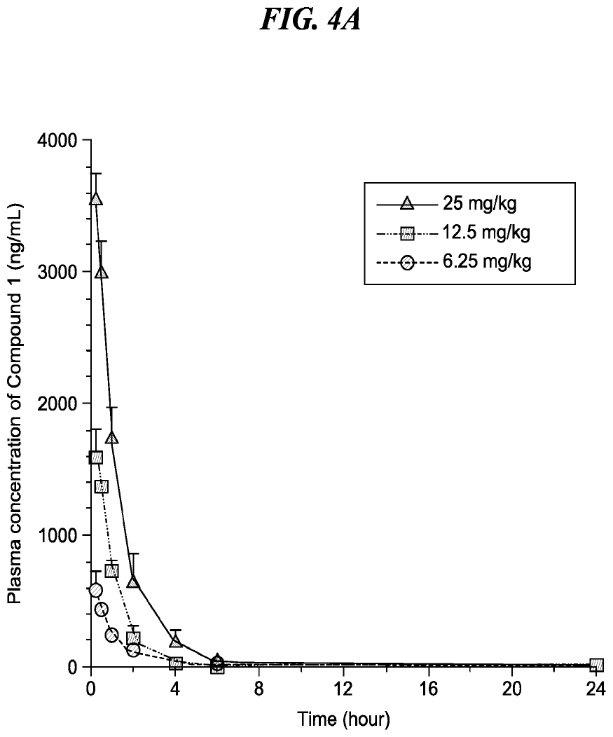

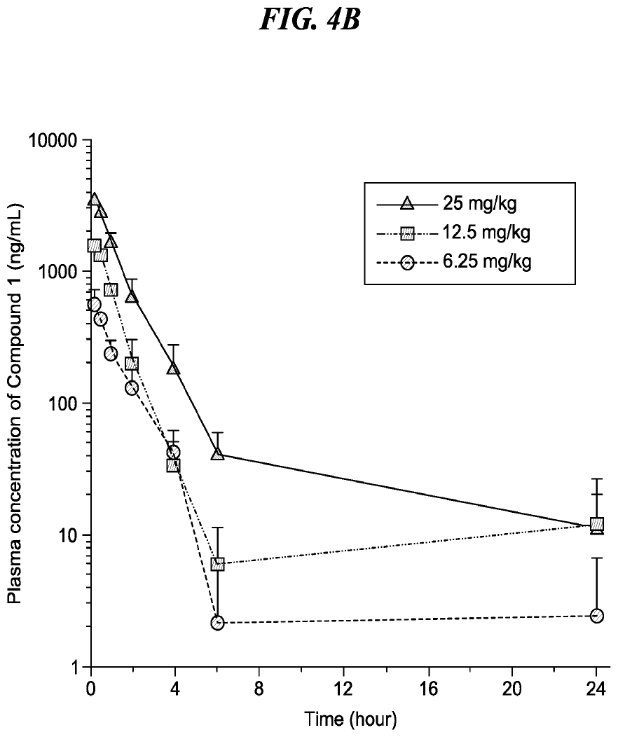

Pharmacokinetics in nude mice after single oral administration. Plasma concentration profiles and PK parameters of Compound (1) were determined in male nude mice following a single oral administration at doses of 6.25, 12.5, and 25 mg/kg (FIGS. 4A and 4B, and Table 4). The Cₘₐₓ, AUCₗₐₛₜ, and AUC₀₋₂₄ values increased in a dose dependent manner.

TABLE 4
Pharmacokinetic parameters of Compound (1) after
single oral administration to male nude mice

| Dose | Tₘₐₓ | Cₘₐₓ | AUCₗₐₛₜ | AUC₀₋₂₄ |
|---|---|---|---|---|
| (mg/kg) | (hr) | (ng/mL) | (ng × hr/mL) | (ng × hr/mL) |
| 6.25 | 0.33 ± 0.14 | 601 ± 111 | 795 ± 229 | 822 ± 208 |
| 12.5 | 0.25 ± 0.00 | 1590 ± 220 | 2010 ± 230 | 2010 ± 230 |
| 25 | 0.25 ± 0.00 | 3580 ± 190 | 5080 ± 820 | 5230 ± 620 |

Abbreviations:
AUCₗₐₛₜ = area under the plasma concentration-time curve from time 0 to the last quantifiable time point;
AUC₀₋₂₄ = area under the plasma concentration-time curve from time 0 to 24 hours;
Cₘₐₓ = maximum plasma concentration;
SD = standard deviation;
Tₘₐₓ = time to reach maximum plasma concentration. Data are shown as the mean ± SD (N = 3/group)

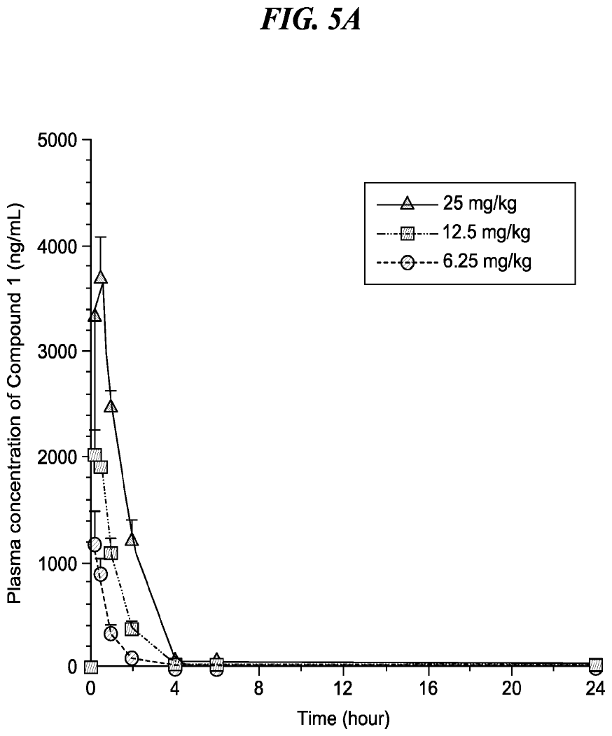

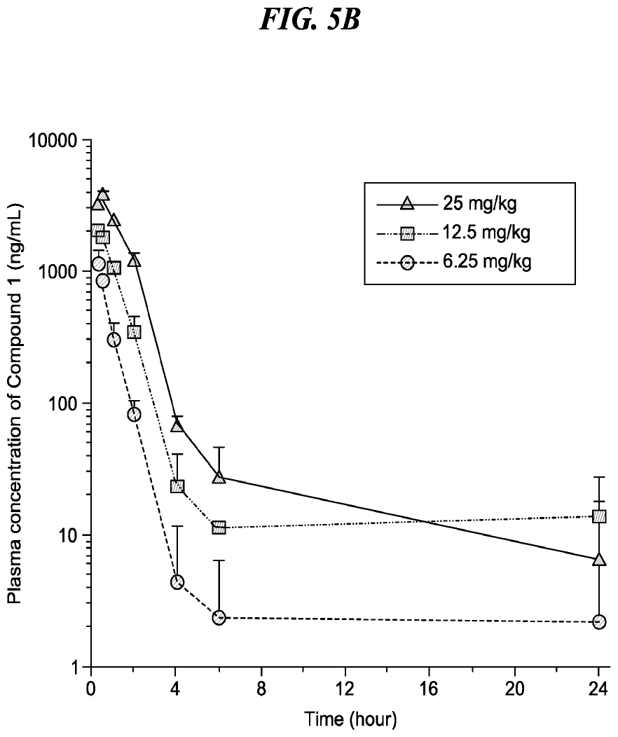

Pharmacokinetics in nude mice after multiple oral administration for 7 days. Plasma concentration profiles and PK parameters of Compound (1) on Day 7 were determined in male nude mice after once daily oral administrations for 7 days at doses of 6.25, 12.5, and 25 mg/kg (FIGS. 5A and 5B, and Table 5). The Cₘₐₓ, AUCₗₐₛₜ, and AUC₀₋₂₄ values increased in a dose-dependent manner.

TABLE 5
Pharmacokinetic parameters of Compound (1) on day 7 after
multiple oral administration for 7 days to male nude mice

| Dose | Tₘₐₓ | Cₘₐₓ | AUCₗₐₛₜ | AUC₀₋₂₄ |
|---|---|---|---|---|
| (mg/kg) | (hr) | (ng/mL) | (ng × hr/mL) | (ng × hr/mL) |
| 6.25 | 0.25 ± 0.00 | 1170 ± 300 | 972 ± 239 | 1030 ± 280 |
| 12.5 | 0.33 ± 0.14 | 2040 ± 230 | 2800 ± 460 | 2830 ± 400 |
| 25 | 0.42 ± 0.14 | 3710 ± 360 | 6180 ± 310 | 6400 ± 430 |

Abbreviations:
AUCₗₐₛₜ = area under the plasma concentration-time curve from time 0 to the last quantifiable time point;
AUC₀₋₂₄ = area under the plasma concentration-time curve from time 0 to 24 hours;
Cₘₐₓ = maximum plasma concentration;
SD = standard deviation;
Tₘₐₓ = time to reach maximum plasma concentration. Data are shown as the mean ± SD (N = 3/group)

Plasma protein binding of Compound (1). Plasma protein binding of Compound (1) in mice, rats, dogs, monkeys, and humans was determined using the equilibrium dialysis method. At Compound (1) concentrations of 0.2, 2, and 20 μmol/L, the plasma protein binding of Compound (1) was 98.8% to 99.5% in mice, 97.3% to 98.9% in rats, 97.4% to 98.0% in dogs, 98.0% to 98.5% in monkeys, and 99.4% to 99.6% in humans, which indicated that the plasma protein binding of Compound (1) was almost constant regardless of Compound (1) concentration. The values of bound fraction of Compound (1) in the solutions containing human serum albumin (HAS), α1-acid glycoprotein (AAG), and γ-globulin (GG) at concentrations between 0.2 and 20 μmol/L ranged from 97.7% to 98.1%, 52.9% to 63.9%, and 40.1% to 53.9%, respectively.

Brain binding of Compound (1) in nude mice. Brain binding of Compound (1) at 0.005, 0.01, 0.05, 0.1, and 0.5 μmol/L using nude mouse brain homogenate was evaluated by equilibrium dialysis method. The brain binding at 0.005 and 0.01 mol/L was not calculated due to the limit of quantification by LC-MS/MS, but at 0.05, 0.1, and 0.5 μmol/L was 99.6±0.0%, 99.5±0.0%, and 99.5±0.0%, respectively. These results indicate that the brain binding of Compound (1) was almost constant regardless of Compound (1) concentration.

Brain penetrability of Compound (1) in nude mice. The brain penetrability of Compound (1) was confirmed by K_{p,brain} values at 0.5 and 1 hour in male nude mice following a single oral administration at 50 mg/kg (Table 6). Based on the brain penetrability study and brain and plasma protein binding studies, K_{p,uu,brain} was calculated to be 0.26±0.05 for 0.5 hour and 0.13±0.03 for 1 hour.

TABLE 6
Plasma and brain concentrations of Compound (1)
after a single oral administration to male mice

| Time | Plasma | Brain | | |
|---|---|---|---|---|
| Point | Concentration | Concentration | | |
| (hr) | (ng/mL) | (ng/g brain) | K_{p, brain} | K_{p, uu, brain} |
| 0.5 | 8720 ± 650 | 3590 ± 600 | 0.415 ± 0.080 | 0.26 ± 0.05 |
| 1 | 6170 ± 910 | 1290 ± 440 | 0.206 ± 0.042 | 0.13 ± 0.03 |

Abbreviations:
K_{p, brain} = brain to plasma concentration ratio;
K_{p, uu, brain} = brain to plasma free concentration ratio;
SD = standard deviation;
Each value represents the mean ± SD (n = 3/time point)

### Pharmacokinetic Drug Interaction

Substrate susceptibility of Compound (1) on human P-gp. Time-dependence of the bidirectional transcellular transport of 1 μmol/L and 10 μmol/L Compound (1) across human P-gp/Lilly Laboratories Cell-Porcine Kidney 1 (LLC-PK1) cells and mock/LLC-PK1 cells was analyzed. Net flux ratios of some samples incubated with 1 μmol/L Compound (1) for 2 or 3 hours was not calculable as concentrations of these samples were below the lower limit of quantification. Therefore, the linearity of cleared volume of these samples could not be determined; however, the net flux ratios of samples incubated with 1 μmol/L Compound (1) for 4 hours with or without 10 μmol/L zosuquidar (which is a human P-gp specific inhibitor) were 1.5±0.6 and 4.7±0.7, respectively, indicating that Compound (1) is a substrate of P-gp. The net flux ratios of samples incubated with 10 μmol/L Compound (1) for 2, 3, and 4 hours were 1.4±0.4 1.7±0.3, and 1.5±0.2, respectively. The net flux ratios samples incubated with 10 μmol/L Compound (1) with 10 μmol/L zosuquidar for 2, 3, and 4 hours were 1.2±0.3, 1.2±0.1, and 1.1±0.1, respectively. These results indicate that the transport of Compound (1) via P-gp is saturable by increases of Compound (1) concentration. The decrease to <2 in net flux ratios with the addition of 10 μmol/L zosuquidar to Compound (1) at 1 μmol/L, and by the increase of Compound (1) concentration from 1 μmol/L to 10 μmol/L, indicate that Compound (1) is a substrate of P-gp.

Inhibitory effect of Compound (1) on human P-gp. Bidirectional transport of [³H]digoxin, which is a typical substrate of human P-gp, for 3 hours in the presence of different concentrations of Compound (1) at 0.01, 0.1, 1, 5, 10, and 100 μmol/L or 10 μmol/L zosuquidar was analyzed. The net flux ratios at the Compound (1) concentrations of 0.01, 0.1, 1, 5, 10, and 100 μmol/L were 5.6±1.0, 5.5±0.7, 6.5±0.5, 2.8±0.5, 1.3±0.2, and 1.0±0.1, respectively. The percent of controls at the Compound (1) concentrations at 0.01, 0.1, 1, 5, 10, and 100 μmol/L were 112%, 110%, 135%, 45%, 7%, and 1%, respectively. Since the percent of controls in the presence of >5 μmol/L Compound (1) were less than 50%, the IC₅₀ value was estimated. Results at 100 μmol/L Compound (1) were excluded from the estimation of IC₅₀ due to its 10-fold higher apparent permeability coefficient compared with that in the group exposed only to [³H]digoxin. The IC₅₀ of Compound (1) was estimated as 3.48 μmol/L. Based on these results, it is concluded that Compound (1) has a potential to inhibit P-gp activity with an estimated IC₅₀ value of 3.48 μmol/L.

Substrate susceptibility of Compound (1) on human BCRP. Time-dependence of the bidirectional transcellular transport of 0.3, 0.5, 1, 3, and 30 μmol/L Compound (1) across human BCRP/Madin-Darby canine kidney II (MDCK II) cells and parent/MDCK II cells was analyzed. Since some samples incubated with 0.3 μmol/L Compound (1) for 2 and/or 3 hours were below the lower limit of quantification, the linearity of cleared volume at 0.3 μmol/L could not be determined; however, the linearity of cleared volume from 2 to 4 hours at 0.5, 1, 3, and 30 μmol/L was confirmed. The net flux ratio of samples incubated with 0.3 μmol/L Compound (1) for 4 hours was 1.5±0.6 and the net flux ratio of samples incubated with 0.3 μmol/L Compound (1) with Ko143 (a typical inhibitor of BCRP) for 4 hours was 1.1±0.2. The net flux ratio at 0.3 μmol/L Compound (1) was less than 2, but flux ratios of samples incubated with 0.3 μmol/L Compound (1) for 3 and 4 hours in parent and human BCRP/MDCK II cells were >2, indicating that Compound (1) was transported by endogenous transporters expressed in MDCK II cells. As transcellular transport of Compound (1) via human BCRP was possibly underestimated due to endogenous transporters in MDCK II cells, these data were not used for the judgment of substrate susceptibility of Compound (1) for human BCRP. The net flux ratios of samples incubated with 0.5 μmol/L Compound (1) were 1.9±0.9 for 2 hours, 3.0±0.9 for 3 hours, and 2.7±0.8 for 4 hours. The net flux ratios of samples incubated with 0.5 μmol/L Compound (1) with 10 μmol/L Ko143, were 1.2±0.3 for 2 hours, 1.2±0.2 for 3 hours, and 1.2±0.2 for 4 hours, respectively. The net flux ratios of samples incubated with 1 μmol/L Compound (1) were 2.3±0.7 for 2 hours, 1.8±0.4 for 3 hours, and 2.2±0.7 for 4 hours, respectively. The net flux ratios of samples incubated with 1 μmol/L Compound (1) with 10 μmol/L Ko143 were 2.0±0.5 for 2 hours, 1.4±0.2 for 3 hours, and 1.4±0.2 for 4 hours, respectively. The net flux ratios of samples incubated with 3 μmol/L Compound (1) were 2.0±0.3 for 2 hours, 1.6±0.3 for 3 hours, and 1.5±0.2 for 4 hours, respectively. The net flux ratios of samples incubated with 30 μmol/L Compound (1) were 1.4±0.3 for 2 hours, 1.1±0.1 for 3 hours, and 1.2±0.1 for 4 hours, respectively. Since several net flux ratios of Compound (1) exceeded 2, which is the criteria for BCRP substrate, Compound (1) is a substrate of BCRP, under the conditions of this assay. Transport of Compound (1) via human BCRP is saturable at >1 μmol/L Compound (1).

Inhibitory effect of Compound (1) on human BCRP. Bidirectional transport of [³H]prazosin (which is a typical substrate of human BCRP) for 3 hours in the presence of different concentrations of Compound (1) at 0.01, 0.1, 0.5, 1, 5, and 10 μmol/L or 10 μmol/L Ko143 was analyzed. The net flux ratio of [³H]prazosin was 4.2±0.6 and Ko143 decreased the net flux ratio of [3H]prazosin to 1.1±0.1. The net flux ratios at each Compound (1) concentration were 4.6±0.4 at 0.01 μmol/L, 4.0±0.3 at 0.1 μmol/L, 2.7±0.2 at 0.5 mol/L, 1.8±0.2 at 1 μmol/L, 1.3±0.2 at 5 μmol/L, and 1.1±0.1 at 10 μmol/L. The percent of control at each Compound (1) concentration were 114% at 0.01 μmol/L, 95% at 0.1 μmol/L, 54% at 0.5 μmol/L, 24% at 1 μmol/L, 10% at 5 μmol/L, and 3% at 10 μmol/L. Since percent of controls at more than 1 μmol/L Compound (1) were below 50%, its IC₅₀ value was estimated as 0.332 μmol/L based on all results. Based on these results, it was concluded that Compound (1) has a potential to inhibit BCRP activity with an IC₅₀ value of 0.332 μmol/L. Since percent of control value with test article at the maximum concentration was below 50%, the IC₅₀ value of Compound (1) against the net flux ratios was estimated by fitting a sigmoidal dose-response curve for the relationship between transporter activity and Compound (1) concentration (four-parameter logistic model) with a nonlinear least square regression analysis. Based on these results, it was concluded that Compound (1) has a potential to inhibit BCRP activity with IC₅₀ value of 0.332 μmol/L.

### Evaluation of the Antitumor Effects of Compound (1) in Nude Mice Bearing Refractory T-Nab/P-Mab NCI-N87 Human Gastric Carcinoma Xenografts

Male nude mice (BALB/cAJcl-mu/mu) were implanted with NCI-N87 human gastric carcinoma (HER2 overexpressing); 1.0×10⁷ cells in 100 μL of PBS were implanted subcutaneously per mouse.

In Step I (days 0-64), the implanted mice were left untreated (control); treated intraperitoneally with trastuzumab (T-mab) once per week at a dosage of 20 mg/kg/day; treated with pertuzumab (P-mab) once per week at a dosage of 20 mg/kg/day; or treated with a combination of T-mab and P-mab once per week at a dosage of 20 mg/kg/day, each. The tumor volumes (TV, mm³) and body weights of the mice were recorded.

In Step II (days 64-79; note day 1 of Step II=day 64 of Step I), the mice which had relapsed from the treatment with the combination of T-mab and P-mab in Step I were divided into three groups. Starting on Day 1 of Step II, the relapsed mice were treated for 14 days with a combination of T-mab and P-mab once per week (day 1 and 8 of Step II) at a dosage of 20 mg/kg/day, each, intraperitoneally; treated with an oral suspension of Compound (1) once per day at a dosage of 12.5 mg/kg/day, per os (PO); or treated with an oral suspension of Compound (1) once per day at a dosage of 25 mg/kg/day. PO. The tumor volumes (TV, mm³) and body weights of the mice were recorded.

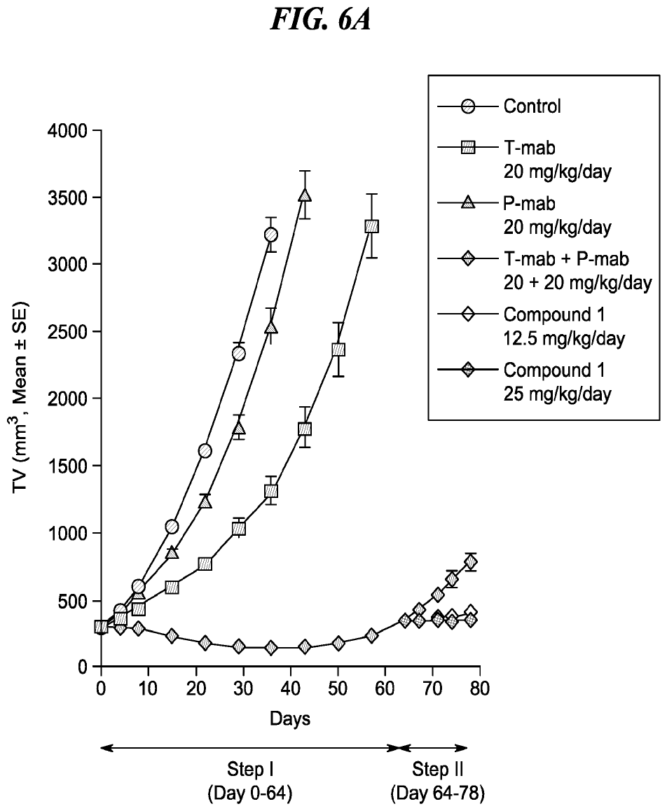

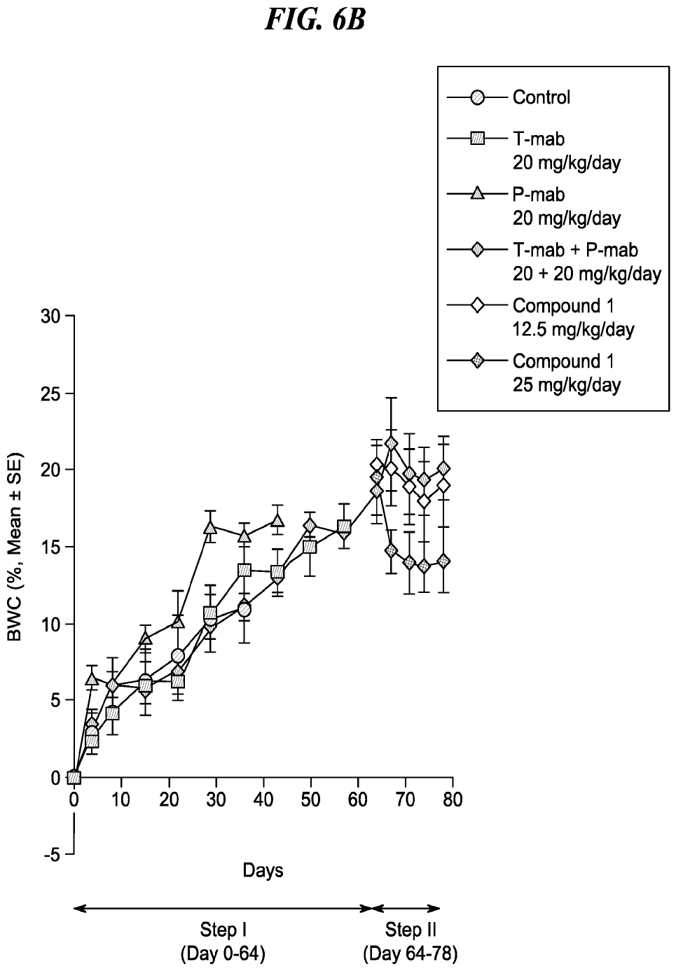

The results are presented in Table 7 (evaluated at Day 22 of Step I), Table 8 (evaluated at Day 15 of Step II), and in FIGS. 6A-6B. As can be seen, continuous exposure to a combination of T-mab and P-mab induced tumor regression in NCI-N87 xenografts initially in Step I, however, tumor regrowth during treatment indicated loss of effectiveness. In tumors with acquired resistance to T-mab and P-mab, Compound (1) was found to be highly efficacious, with Compound (1) providing T/C values of 52.5% and 46.1%, for 12.5 and 25 mg/kg/day dosages, respectively, relative to the combination of T-mab/P-mab over the same time frame. Further, Compound (1) did not cause severe body weight loss at either dosage, signifying acceptable tolerance.

TABLE 7
Refractory T-mab/P-mab Model Step I (Day 22)

| | | | | | | P-value | P-value | P-value | | | |
|---|---|---|---|---|---|---|---|---|---|---|---|
| | | | | | | vs. | vs. | vs. | | | |
| | | | | | TV^{a)} | Control | T-mab | P-mab | | | |
| | | | Number | Number | (mm³, | by | by Aspin- | by Aspin- | | RTV^{c)} | BWC^{d)} |
| | Dose | Treatment | of | of | mean ± | Dunnett's | Welch's | Welch's | T/C^{b)} | (mean ± | (mean ± |
| Drug | (mg/kg/day) | (ip) | Animals | Death | SE) | test | t-test | t-test | (%) | SE) | SE) |
| Control | — | — | 5 | 0 | 1612.6 ± | — | — | — | 100.0 | 5.2 ± | 7.90 ± |
| (Non- | | | | | 43.4 | | | | | 0.2 | 2.60 |
| treatment) | | | | | | | | | | | |
| T-mab | 20 | Days 1, | 5 | 0 | 772.9 ± | <0.0001 | — | — | 47.9 | 2.5 ± | 6.30 ± |
| | | 8, 15(I) | | | 37.9 | | | | | 0.1 | 1.20 |
| P-mab | 20 | Days 1, | 5 | 0 | 1233.1 ± | 0.0005 | — | — | 76.5 | 4.0 ± | 10.20 ± |
| | | 8, 15(I) | | | 45.5 | | | | | 0.2 | 2.00 |
| T-mab + | 20 + 20 | Days 1, | 50 | 0 | 170.8 ± | <0.0001 | <0.0001 | <0.0001 | 10.6 | 0.6 ± | 7.00 ± |
| P-mab | | 8, 15(I) | | | 4.4 | | | | | 0.0 | 0.50 |

^{a)}Tumor volume (TV) was calculated on day 22 (I) according to: TV = (length) × (width) × (width)/2
^{b)}Treatment/Control (T/C) on day 22 (Step I) on the basis of RTV was calculated as: T/C (%) = (mean RTV in the administration group)/(mean RTV in the control group) × 100
^{c)}Relative tumor volume (RTV) was calculated as the ratio of TV on Day 22 (Step I) to that on Day 0 (Step I)
^{d)}Body weight change (BWC) was calculated as: BWC (%) = [(body weight at Day 22 Step I) − (body weight at Day 0 Step I)]/(body weight at Day 0 Step I) × 100
ip: intraperitoneal
SE: standard error

TABLE 8
Refractory T-mab/P-mab Model Step II (Day 15)

| | | | | | | P-value | | | |
|---|---|---|---|---|---|---|---|---|---|
| | | | | | | vs. | | | |
| | | | | | | T-mab + | | | |
| | | | | | TV^{a)} | P-mab | | | |
| | | | Number | Number | (mm³, | by | | RTV^{d)} | BWC^{e)} |
| | Dose | | of | of | mean ± | Dunnett's | T/C^{c)} | (mean ± | (mean ± |
| Drug | (mg/kg/day) | Treatment | Animals | Death | SE) | test | (%) | SE) | SE) |
| T-mab + | 20 + 20 | Days 1, | 8 | 0 | 785.9 ± | — | 100.0 | 2.2 ± | 1.3 ± |
| P-mab | | 8(II), ip | | | 64.2 | | | 0.1 | 1.0 |
| Compound (1) | 12.5^{b)} | Days 1- | 8 | 0 | 412.5 ± | <0.0001 | 52.5 | 1.2 ± | −1.2 ± |
| | | 14(II), po | | | 28.5 | | | 0.0 | 1.3 |
| Compound (1) | 25^{b)} | Days 1- | 8 | 0 | 362.4 ± | <0.0001 | 46.1 | 1.0 ± | −4.4 ± |
| | | 14(II), po | | | 19.6 | | | 0.0 | 1.4 |

^{a)}Tumor volume (TV) on day 15 (Step II) was calculated according to: TV = (length) × (width) × (width)/2
^{b)}Administered as a fumarate salt; dosages based on amount of free form
^{c)}Treatment/Control (T/C) on day 15 (Step II) on the basis of RTV was calculated as: T/C (%) = (mean RTV in the administration group)/(mean RTV in the T-mab + P-mab administration group) × 100
^{d)}Relative tumor volume (RTV) was calculated as the ratio of TV on Day 15 (Step II) to that on Day 1 (Step II)
^{e)}Body weight change (BWC) was calculated as: BWC (%) = [(body weight at Day 15 Step II) − (body weight at Day 1 Step II)]/body weight at Day 1 Step II) × 100
ip: intraperitoneal
po: per os
SE: standard error

### Evaluation of the Antitumor Effects of Compound (1) in Nude Mice Bearing Refractory T-DM1 NCI-N87 Human Gastric Carcinoma Xenografts

Male nude mice (BALB/cAJ1-mu/mu) were implanted with NCI-N87 human gastric carcinoma (HER2 overexpressing); 1.0×10⁷ cells in 100 μL of PBS were implanted subcutaneously per mouse.

In Step I (days 0-85), the implanted mice were left untreated (control); or treated with intravenous trastuzumab emtansine (T-DM1) once every 3 weeks at a dosage of 10 mg/kg/day.

In Step II (days 85-106; note day 1 of Step II=day 85 of Step I), the mice which had relapsed from the treatment with intravenous trastuzumab emtansine in Step I were divided into three groups. Starting on Day 1 of Step II, the relapsed mice were treated for 21 days with intravenous trastuzumab emtansine (single dose on day 1 of Step II) at a dosage of 10 mg/kg/day; treated with an oral suspension of Compound (1) once per day at a dosage of 12.5 mg/kg/day. PO; or treated with an oral suspension of Compound (1) once per day at a dosage of 25 mg/kg/day, PO. The tumor volumes (TV, mm³) and body weights of the mice were recorded.

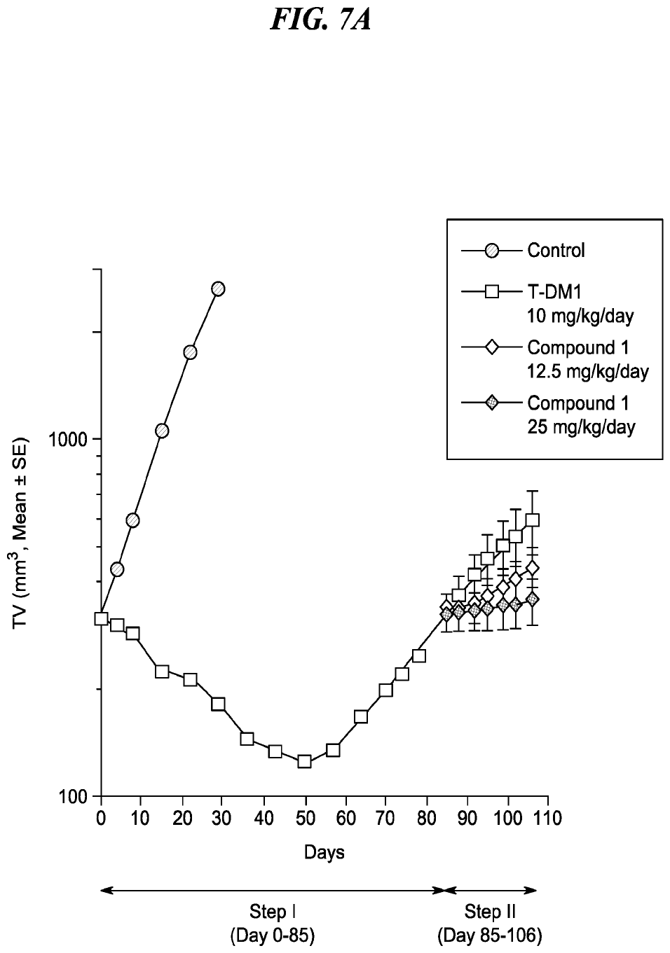

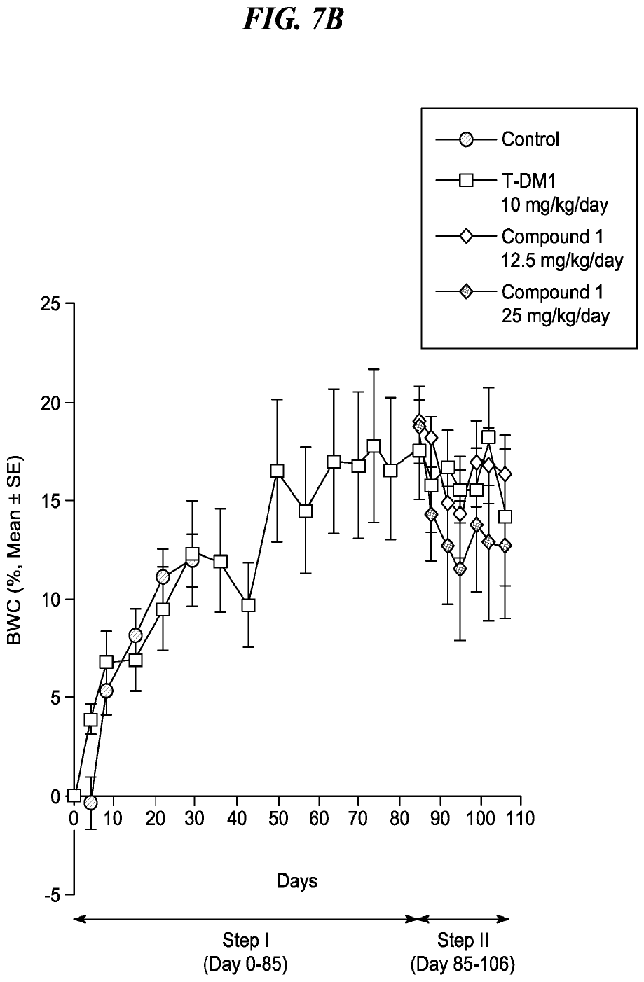

The results are presented in Table 9 (evaluated at Day 22 of Step II), and in FIGS. 7A-7B. As can be seen, continuous exposure to T-DM1 induced tumor regression in NCI-N87 xenografts initially, however tumor regrowth during treatment indicated loss of effectiveness. In tumors with acquired resistance to T-DM1, Compound (1) was found to be efficacious, providing T/C values of 75.7% and 62.4%, for 12.5 and 25 mg/kg/day dosages, respectively, relative to T-DM1 over the same time frame. Further, Compound (1) did not cause severe body weight loss at either dosage, signifying acceptable tolerance.

TABLE 9
Refractory T-DM1 Model Step II (Day 22)

| | | | | | | | P-value | |
|---|---|---|---|---|---|---|---|---|
| | | | | | | | vs. | |
| | | | | | TV^{a)} | RTV^{c)} | T-DM1 | |
| | | | Number | Number | (mm³, | (mm³, | (by | |
| | Dose | | of | of | mean ± | mean ± | Dunnett's | T/C^{d)} |
| Drug | (mg/kg/day) | Treatment | Animals | Death | SE) | SE) | test) | (%) |
| T-DM1 | 10 | Day | 7 | 0 | 595.1 ± | 1.73 ± | — | 100.0 |
| | | 1(II), iv | | | 120.9 | 0.16 | | |
| Compound | 12.5^{b)} | Days 1- | 7 | 0 | 435.7 ± | 1.31 ± | 0.0408 | 75.7 |
| (1) | | 21(II), po | | | 58.8 | 0.08 | | |
| Compound | 25^{b)} | Days 1- | 7 | 0 | 353.7 ± | 1.08 ± | 0.0007 | 62.4 |
| (1) | | 21(II), po | | | 51.1 | 0.09 | | |

^{a)}Tumor volume (TV) on day 22 (Step II) was calculated according to: TV = (length) × (width) × (width)/2
^{b)}Administered as a fumarate salt; dosages based on amount of free form
^{c)}Relative tumor volume (RTV) was calculated as the ratio of TV on Day 22 (Step II) to that on Day 1 (Step II)
^{d)}Treatment/Control (T/C) on day 22 (Step II) on the basis of RTV was calculated as: T/C (%) = (RTV in the administration group)/(RTV in the T-DM1 administration group) × 100
iv: intravenous
po: per os
SE: standard error

### Evaluation of the Antitumor Effects of Compound (1) in Nude Mice Bearing Refractory T-DM1 NCI-N87 Human Gastric Carcinoma Xenografts: Comparison to Lapatinib and Tucatinib

Refractory T-DM1 NCI-N87 human gastric carcinoma tumors (HER2 overexpressing) were obtained by continuous exposure to intravenous trastuzumab emtansine (T-DM1) once every 3 weeks at a dosage of 10 mg/kg/day for 126 days, according to the procedure described in Irie H, Kawabata R, Fujioka Y, Nakagawa F, Itadani H. Nagase H, Ito K, Uchida J, Ohkubo S, Matsuo K. Acquired resistance to trastuzumab/pertuzumab or to T-DM1 in vivo can be overcome by HER2 kinase inhibition with TAS0728. Cancer Sci. 2020 June; 111(6):2123-2131. The NCI-N87 tumor fragment #6 obtained from the above procedure was then passaged two times into male nude mice (BALB/cAJcl-nu/nu) subcutaneously before the efficacy test of Compound (1).

Starting on day 1 after tumor passaging and continuing for 21 days, the tumor fragment-implanted mice were left untreated (control); treated with intravenous trastuzumab emtansine (T-DM1) at a dosage of 10 mg/kg/day (single dose on day 1 after tumor passaging); treated with lapatinib twice per day (b.i.d.) at 50 mg/kg each dose, PO; treated with tucatinib twice per day (b.i.d.) at 75 mg/kg each dose, PO; treated with an oral suspension of Compound (1) once per day at a dosage of 12.5 mg/kg/day, PO; or treated with an oral suspension of Compound (1) once per day at a dosage of 20 mg/kg/day, PO. The tumor volumes (TV, mnm³) and body weights of the mice were recorded.

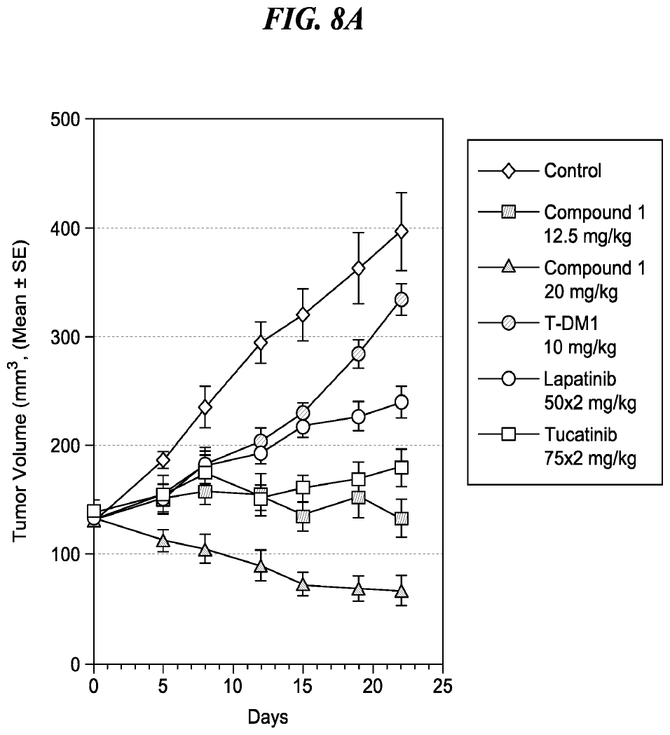

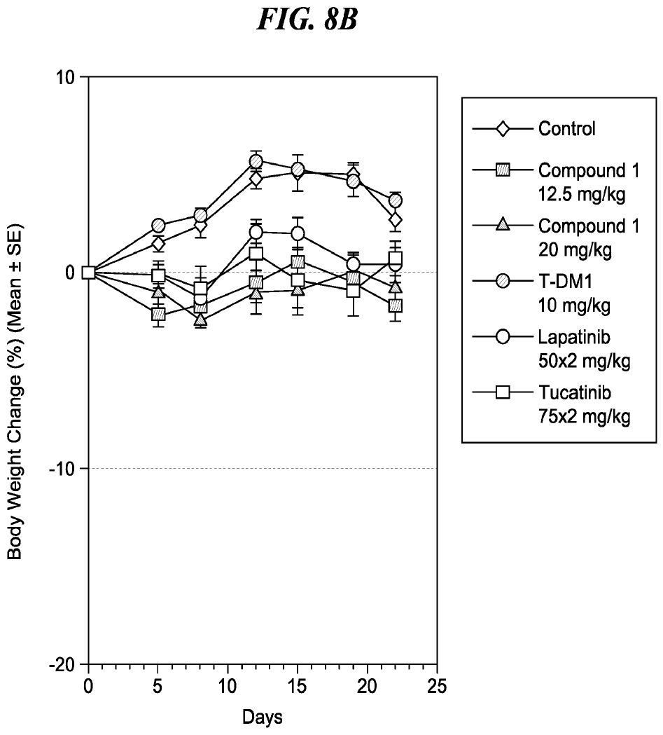

The results are presented in FIGS. 8A-8B. As can be seen, Compound (1) was found to be highly efficacious against T-DM1 refractory tumors, arresting tumor growth at the lower dose and inducing tumor regression at the higher dosage of 20 mg/kg. Notably, despite lower dosages, Compound (1) was found to provide superior antitumor effects compared to treatment using either lapatinib or tucatinib. Further, Compound (1) did not cause body weight loss at either the 12.5 or 20 mg/kg dosage, signifying acceptable tolerance.

### Evaluation of the Antitumor Effects of Compound (1) in Nude Mice Bearing Refractory T-DM1 NCI-N87 Human Gastric Carcinoma Xenografts: Comparison to Trastuzumab Deruxtecan (DS-8201a)

Refractory T-DM1 NCI-N87 human gastric carcinoma tumor fragments #6 were obtained in the same manner as described above, and then the monoclonal cell line, NCI-N87 tumor fragment #6, clone 3 was isolated by passages in vitro. The obtained cell line was implanted into BALB/cAJcl-mu/nu male mice (8.0×10⁶ cells in 100 μL of PBS/mouse, subcutaneous) for the efficacy test.

Starting on Day 1 after tumor implanting and continuing for 21 days, the implanted mice were left untreated (control); treated with intravenous trastuzumab emtansine (T-DM1) at a dosage of 10 mg/kg/day (single dose on day 1 after tumor implanting); treated with intravenous trastuzumab deruxtecan (DS-8201a) at a dosage of 1 mg/kg/day (single dose on day 1 after tumor implanting); treated with intravenous trastuzumab deruxtecan (DS-8201a) at a dosage of 10 mg/kg/day (single dose on day 1 after tumor implanting); treated with an oral suspension of Compound (1) once per day at a dosage of 12.5 mg/kg/day, PO; or treated with an oral suspension of Compound (1) once per day at a dosage of 20 mg/kg/day, PO. The tumor volumes (TV, mm) and body weights of the mice were recorded.

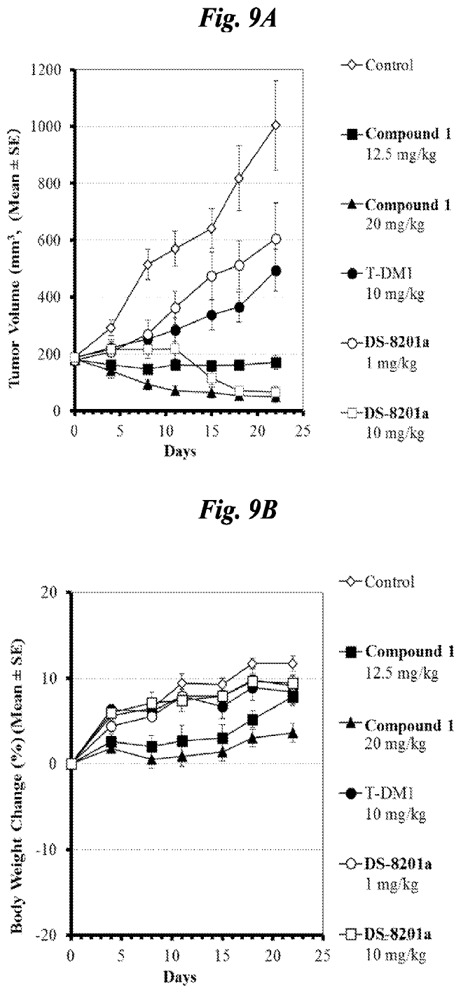

The results are presented in FIGS. 9A-9B. DS-8201a has been previously shown to be highly active in vivo in HER2-positive mouse models using NCI-N87 parental cell xenografts, inducing tumor growth inhibition in a dose-dependent manner and tumor regression with a single dosing of 1 mg/kg or more, with a single dose of 4 mg/kg providing 99% tumor growth inhibition. See Yusuke Ogitani, Tetsuo Aida, Katsunobu Hagihara et al. DS-8201a, A Novel HER2-Targeting ADC with a Novel DNA Topoisomerase I Inhibitor, Demonstrates a Promising Antitumor Efficacy with Differentiation from T-DM1, Clin. Cancer Res. Oct. 15, 2016; 22(20):5097-5108. However, as seen in FIG. 9A, DS-8201a showed much weaker efficacy in T-DM1 resistant cells (using NCI-N87 tumor fragment #6, clone 3), with a single dose of 1 mg/kg failing to inhibit tumor growth, similar to T-DM1. Rather, a dosing of 10 mg/kg of antibody-drug conjugate DS-8201a was needed to achieve tumor regression in this refractory tumor model. On the other hand, Compound (1) was found to be highly efficacious against T-DM1 refractory tumors, arresting tumor growth at the lower dose and inducing tumor regression at the higher dosage of 20 mg/kg. Compound (1) was also found to be well tolerated and did not cause body weight loss at either the 12.5 or 20 mg/kg dosage.

### Evaluation of the Growth Inhibitory Effect of Compound (1) in NRG1 Fusion-Driven Cancer Cell Line

To investigate the inhibitory effect of Compound (1) on the proliferation of NRG1 fusion-driven cancer, the 50% growth inhibitory concentration (GI₅₀) value was determined for MDA-MB-175VII cells (human breast ductal carcinoma which expresses the DOC4-NRG1 fusion protein). GI₅₀ values were also determined for currently approved EGFR/HER2 tyrosine kinase inhibitors: afatinib, osimertinib, tucatinib, lapatinib, and neratinib, and one investigational drug (poziotinib). Mean values were determined from 3-4 independent experiments.

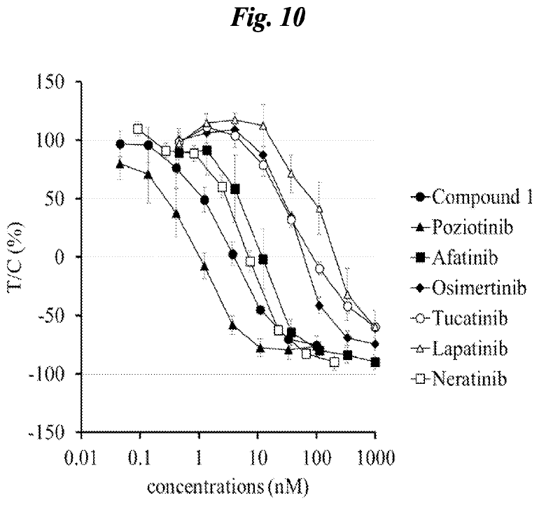

Mean GI₅₀ results are presented in Table 10 and shown in FIG. 10. Compound (1) monofumarate salt was found to be highly potent against MDA-MB-175VII with a mean GI₅₀ value of 1.2±0.3 nM. For comparison, Compound (1) was found to be over twice as potent against MDA-MB-175VII as the next most active EGFR/HER2 TKI that is currently approved, neratinib, which provided a mean GI₅₀ of 3.0±0.6 nM. Other approved EGFR/HER2 TKIs were found to be less potent with mean G1₅₀ values for afatinib, osimertinib, tucatinib, and lapatinib of 5.6±2.7 nM, 29.4±1.5 nM, 27.6±5.5 nM, and 89.5±39.5 nM, respectively. Investigational drug poziotinib was also found to be highly effective with a mean GI₅₀ value of 0.3±0.2 nM. These results also demonstrate that it is difficult to predict which EGFR/HER2 inhibitors will be active in cancers with aberrant activated EGFR and/or HER2 as a result of NRG1 fusions.

TABLE 10
GI₅₀ (nM) values against MDA-MB-
175VII (harboring DOC4-NRG1 fusion)

| | mean | SD |
|---|---|---|
| Compound (1) | 1.2 | 0.3 |
| Poziotinib | 0.3 | 0.2 |
| Afatinib | 5.6 | 2.7 |
| Osimertinib | 29.4 | 1.5 |
| Tucatinib | 27.6 | 5.5 |
| Lapatinib | 89.5 | 39.5 |
| Neratinib | 3.0 | 0.6 |

### Evaluation of the Antitumor Effects of Compound (1) in Nude Mice Bearing Intracranial NCI-N87-Luc Human Gastric Carcinoma Xenografts

The intracranial antitumor effect of Compound (1) with various dosing schedules ((i) QD, (ii) intermittent, and (iii) 4 days-on/3 days-off) was evaluated using Compound (1) in nude mice intracranially implanted with NCI-N87-luc cells, which were engineered to stably express the luciferase gene using the HER2-overexpressing NCI-N87 human gastric carcinoma cell line.

Compound (1) dosing suspensions using 0.5% weight per volume (w/v) hydroxypropyl methylcellulose (HPMC) as vehicle were prepared with Compound (1) at 2 dose levels (25.0 or 50.0 mg/kg/day) and orally administered with various dosing schedules ((i) QD, (ii) intermittent, and (iii) 4 days-on/3 days-off) for 21 days. The (i) QD dosing schedule in this example was dosing every day on day 1 to day 21. The (ii) intermittent dosing schedule in this example used sequential 7-day periods that each comprise alternating 4 days-on and 3 days-off. That is, this schedule involved dosing on days 1, 3, 5, 7, 8, 10, 12, 14, 15, 17, 19, and 21. The (iii) 4 days-on/3 days-off dosing schedule was dosing on days 1, 2, 3, 4, 8, 9, 10, 11, 15, 16, 17, and 18. The control group received 0.5% w/v HPMC solution orally once daily for 21 days.

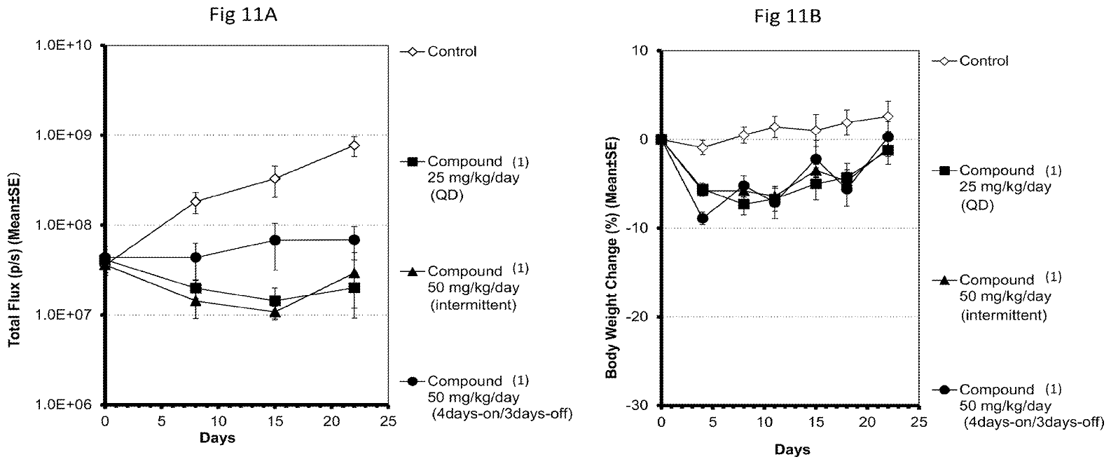

At the evaluation on Day 22, all groups treated with Compound (1) which were orally administered with various dosing schedules ((i), (ii), and (iii)) for 21 days showed significant tumor growth inhibition compared with the control group (P<0.05, Dunnett's test) (FIG. 11A).

The T/C ratios of the mean total flux in the groups treated with Compound (1) were 2.6% for (i) at 25.0 mg/kg/day, 3.8% for (ii) at 50.0 mg/kg/day, and 8.9% for (iii) at 50.0 mg/kg/day for 21 days. No mice had >20% loss in body weight from Day 0 during the treatment period. For the groups treated with Compound (1), mean body weight changes on Day 22 were −1.2% for (i) at 25.0 mg/kg/day, −1.0% for (ii) at 50.0 mg/kg/day, and 0.3% for (iii) at 50.0 mg/kg/day for 21 days.

These findings indicated that Compound (1) showed intracranial antitumor activity without severe body weight loss in nude mice implanted with NCI-N87-luc human gastric carcinoma in the brain parenchyma, with various dosing schedules ((i), (ii), and (iii)).

Obviously, numerous modifications and variations of the present invention are possible in light of the above teachings. It is therefore to be understood that within the scope of the appended claims, the invention may be practiced otherwise than as specifically described herein.

1. A method of treating a subject with a cancer having at least one aberration in EGFR and/or HER2, the method comprising:
administering to the subject an effective amount of 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

2. The method of claim 1, wherein the cancer is a solid tumor.

3. The method of claim 1, wherein the cancer is at least one selected from the group consisting of lung cancer, breast cancer, gastric cancer, bladder cancer, and biliary cancer.

4. The method of claim 1, wherein the cancer has metastasized to the brain of the subject.

5. The method of claim 1, wherein the cancer has an EGFR amplification/overexpression and/or a HER2 amplification/overexpression.

6. The method of claim 1, wherein the cancer has an EGFR mutation and/or a HER2 mutation.

7. The method of claim 1, wherein the cancer has an EGFR exon 20 insertion mutation and/or a HER2 exon 20 insertion mutation.

8. The method of claim 7, wherein the cancer has an EGFR exon 20 insertion mutation.

9. The method of claim 7, wherein the cancer has a HER2 exon 20 insertion mutation.

10. The method of claim 7, wherein the cancer has metastasized to the brain of the subject.

11. The method of claim 7, wherein the cancer is non-small cell lung cancer.

12. The method of claim 1, wherein the cancer has an EGFR amplification and/or an EGFRvIII mutation.

13. The method of claim 12, wherein the cancer is glioblastoma.

14. The method of claim 1, wherein the cancer having at least one aberration in EGFR and/or HER2 is a cancer harboring an aberration in NRG1.

15. The method of claim 14, wherein the cancer harboring an aberration in NRG1 is an NRG1 fusion-driven cancer harboring an NRG1 fusion.

16. The method of claim 14, wherein the cancer harboring an aberration in NRG1 is at least one selected from the group consisting of lung cancer, breast cancer, colorectal cancer, pancreatic cancer, biliary cancer, ovarian cancer, and gastric cancer.

17. The method of claim 15, wherein the NRG1 fusion-driven cancer is at least one selected from the group consisting of lung cancer, breast cancer, colorectal cancer, pancreatic cancer, biliary cancer, and ovarian cancer.

18. The method of claim 1, wherein the cancer is unresectable.

19. The method of claim 1, wherein the cancer is a recurrent or refractory cancer.

20. The method of claim 1, wherein the cancer is a recurrent or refractory HER2-positive cancer.

21. The method of claim 20, wherein the subject with the recurrent or refractory HER2-positive cancer has previously undergone a treatment regimen with a HER2 inhibitor prior to administering 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

22. The method of claim 21, wherein the recurrent or refractory HER2-positive cancer acquired resistance to or intractability from the treatment regimen with the HER2 inhibitor.

23. The method of claim 21, wherein the HER2 inhibitor is a HER2 tyrosine kinase inhibitor.

24. The method of claim 23, wherein the HER2 tyrosine kinase inhibitor is at least one selected from the group consisting of afatinib, lapatinib, neratinib, and tucatinib.

25. The method of claim 21, wherein the HER2 inhibitor is an anti-HER2 antibody or drug conjugate thereof.

26. The method of claim 25, wherein the anti-HER2 antibody or drug conjugate thereof is at least one selected from the group consisting of trastuzumab, trastuzumab emtansine, pertuzumab, margetuximab, and trastuzumab deruxtecan.

27. The method of claim 21, wherein the subject with the recurrent or refractory HER2-positive cancer has previously undergone at least two treatment regimens with at least two different HER2 inhibitors prior to administering 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

28. The method of claim 20, wherein the recurrent or refractory HER2-positive cancer is recurrent or refractory HER2-positive breast cancer or recurrent or refractory HER2-positive gastric cancer.

29. The method of claim 1, wherein the subject is determined to have the aberration in EGFR and/or HER2 prior to administering 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof.

30. The method of claim 1, wherein the 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered orally to the subject.

31. The method of claim 1, wherein the 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered to the subject once per day (QD).

32. The method of claim 1, wherein the 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered to the subject twice per day (BID).

33. The method of claim 1, wherein from about 5 to about 480 mg of 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered to the subject per day.

34. The method of claim 1, wherein about 15 to about 240 mg of 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered to the subject per day.

35. The method of claim 1, wherein the 7-((3R,5S)-1-acryloyl-5-methylpyrrolidin-3-yl)-4-amino-6-(cyclopropylethynyl)-N—((R)-1-phenylethyl)-7H-pyrrolo[2,3-d]pyrimidine-5-carboxamide or a pharmaceutically acceptable salt thereof is administered daily to the subject for at least 28 days.
